(19) United States
(12) Patent Application Publication
(10) Pub. No.: US 2013/0344569 A1
(43) Pub. Date: Dec. 26, 2013

(54) PERMANENT HUMAN CELL LINES FOR THE PRODUCTION OF INFLUENZA VIRUSES

(71) Applicant: Gudrun Schiedner, Cologne (DE)

(72) Inventor: Gudrun Schiedner, Cologne (DE)

(73) Assignee: CEVEC PHARMACEUTICALS GMBH, Cologne (DE)

(21) Appl. No.: 13/816,976

(22) PCT Filed: Aug. 16, 2011
(86) PCT No.: PCT/DE2011/075194

§ 371 Date: May 6, 2013

(30) Foreign Application Priority Data
Aug. 16, 2010 (DE) DE 102010037008.8
May 13, 2011 (DE) DE 102011050353.6

(51) Int. Cl. C12N 7/00

(52) U.S. Cl. CPC C12N 7/00
USPC 435/235.1

(57) ABSTRACT

The present invention relates to a method for the production of an influenza virus-based vaccine using permanent human amniocyte cells, as well as the use of a permanent human amniocyte cell for the production of a influenza virus-based vaccine.

This application is a national phase application under 35 U.S.C. §371 of International Application No. PCT/DE2011/075194, filed Mar. 15, 2007, which claims priority to German Application No. DE 10 2010 037 008.8, filed Aug. 16, 2010 and German Application No. DE 10 2011 050 353.6, filed May 13, 2011. The entire text of each of the above referenced disclosures is specifically incorporated herein by reference.

The present invention relates to a method for the production of an influenza virus based vaccine using permanent human amniocyte cells, as well as to the use of a permanent human amniocyte cell for the production of a influenza virus based vaccine.

The vaccination is the most important measure in health care, to prevent illness caused by the annual influenza epidemic. The successful use of vaccines is dependent on the quickest-possible production of sufficiently large amounts of vaccines, such as killed viruses, from stable and easy-to-use sources. The rapid development of vaccines and their adequate availability are crucial in the fight against many human and animal diseases. As a result of delays in the production of vaccines and quantitative loss, problems in the handling of outbreaks of disease may occur. This resulted in the recent efforts to focus on the cultivation of viruses in cell culture for the use as vaccines.

So far, the available influenza vaccines are produced in embryonated chicken eggs. These chicken eggs must have been shown to be free of certain viral and bacterial contamination. These so-called “specific pathogen free” (SPF) chicken eggs are commercially available. Even though chicken eggs have been found to be very useful in the propagation of animal and human viruses, they bear some disadvantages in the production of vaccines. For example, in the event of a pandemic, there will be a high demand for chicken eggs for vaccine production since one egg is needed for the production of one dose of a conventional vaccine. Given the limited availability of chicken eggs a period of about a year should be expected to provide the chicken eggs in sufficient quantity. Further, there are also influenza subtypes that are highly pathogenic for chickens, so that they may cause a shortage of supply of chicken eggs in case of a pandemic. In addition, the production process is very cost-intensive and time-consuming. Another disadvantage of vaccine production in chicken eggs is that these vaccines are usually not free of chicken egg white, and thus in some patients allergic reactions may occur. Last but not least, the possible selection of subpopulation differing from the naturally occurring virus requires alternative host cell systems.

Contrary to chicken eggs, cells for influenza vaccine production on cell culture basis are always available. They are stored deep-frozen and may be thawed quickly and reproduced in the required amount at any time on demand. Thus, the vaccine production can be started at any desired time. In the event of unexpectedly high demand, or when unexpected new strains of the virus circulate more frequently, an appropriate vaccine may be provided in a short time.

The production process using cell culture enables the production of viruses as vaccines in a closed, standardized system under defined, controlled conditions. Due to the controlled production method, the finished flu vaccine requires no addition of antibiotics. Since the preparation of cell culture influenza vaccines is completely independent of chicken eggs, the vaccine produced in this manner is without chicken white egg and thus, can not cause allergic reactions due to intolerance of chicken egg white in patients.

Presently, mainly the three cell lines, namely the human PER.C6 cells, the Madin Darby Canine Kidney (MDCK) cells, and guenon kidney cells (Vero) are used for influenza vaccine production. In addition, currently a duck retina cell line (AGE1.CR) and avian embryonic stem cell lines are developed. The production of vaccines in mammalian cells does represent an alternative to the chicken-egg-based vaccine production, however, these cells require serum and/or the attachment to a solid support for their growth. This makes it difficult and therefore more expensive to produce vaccines in these cells, since, for safety reasons, the serum has to be separated completely and the growth on solid supports is limited, thus leading to lower yields.

An advantage of the vaccine production in mammalian cells is that the isolation and replication of the virus in the cell culture do not generate any passenger-dependent selection of a phenotype differing from the clinical wild-type. Therefore, the viral glycoprotein hemagglutinin, by means of which the attachment to the cell to be infected and the integration of the virus into the cell occurs, is expressed as a native form, and thereby, it has an improved specificity and avidity and thus, enables a cell-mediated immunity in people.

Thus, the object of the invention is to provide improved permanent human cell lines for the production of influenza virus based vaccines.

The object is solved by the subject matter as defined in the claims.

The figures illustrate the invention.

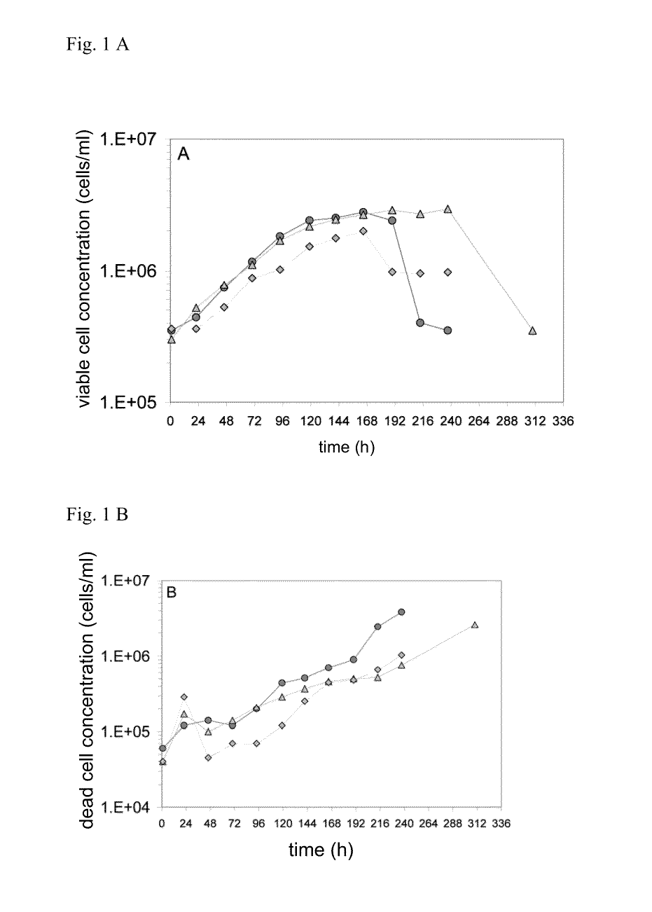

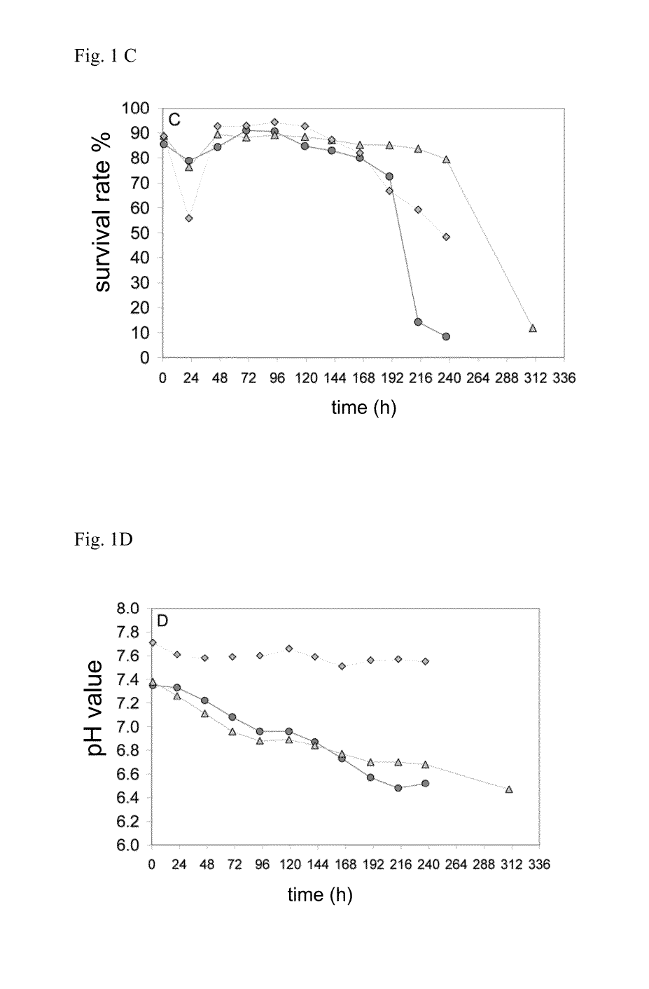

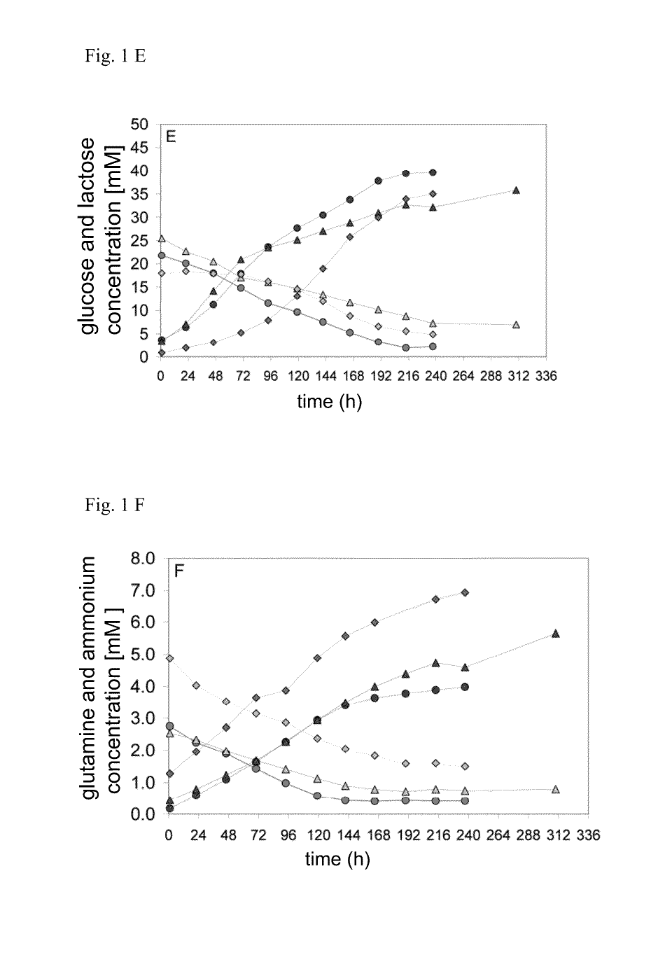

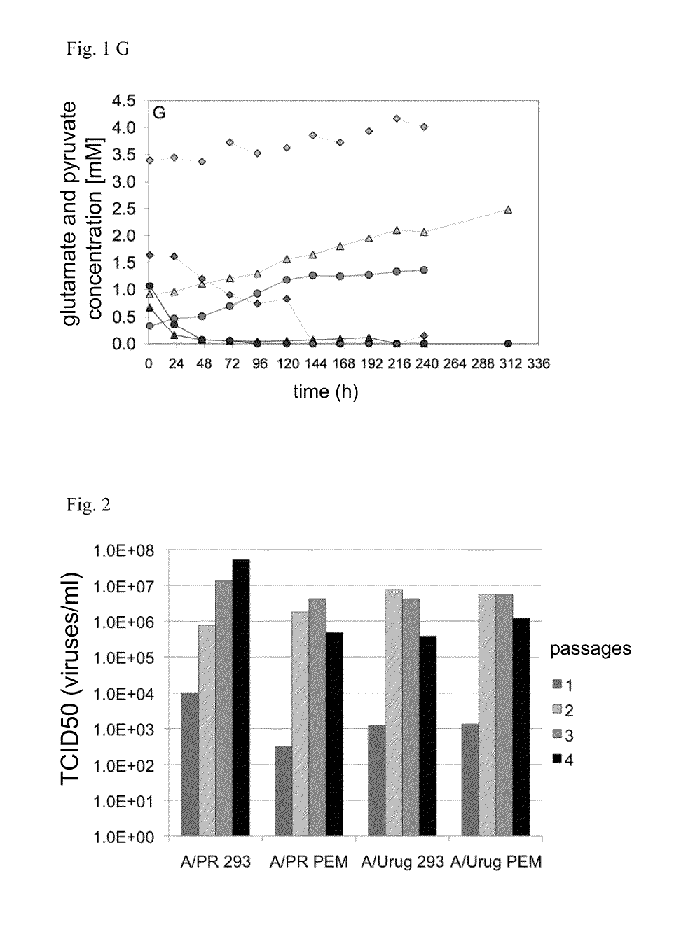

FIG. 1A to G shows schematically the course of different parameters during the cultivation of the permanent amniocyte cell line CAP 1D5 in 293SFMII medium (), of the permanent amniocyte cell line CAP 1D5 in PEM medium (▴) and the permanent canine kidney cell line MDCK.SUS2 (Madin Darby Canine Kidney) in SMIF8 medium (♦) in 100 ml shake flasks. FIG. 1A graphically shows the course of the viable cell concentration of the three cell lines in comparison; FIG. 1B shows the course of the dead cell concentration of the cell lines; and FIG. 1C shows the course of the survival rate of the cell lines. FIGS. 1D to G schematically show the course of the pH value (D), the glucose (bright symbols) and lactose (dark symbols) concentration (E), glutamine (Gln) (bright symbols) and ammonium (dark symbols) concentration (F), and the glutamic acid (Glu) (bright symbols) and pyruvate (dark symbols) concentration (G).

FIG. 2 shows a bar graph depicting the measured virus titers as the TCID₅₀ value over 4 passages of the influenza strains A/PR/8/34 (H1N1) and A/Uruguay/716/2007 (H3N2) in CAP-1D5 cells 293SFMII and PEM medium. Abbreviations: A/PR 293: influenza strain A/PR/8/34 (H1N1) in CAP-1D5 cells in 293SFMII medium, A/PR PEM: influenza strain A/PR/8/34 (H1N1) in CAP-1D5 cells in PEM medium; A/Urug 293: A/Uruguay/716/2007 strain of influenza (H3N2) in CAP-1D5 cells in 293SFMII medium; A/Urug PEM: influenza strain A/Uruguay/716/2007 (H3N2) in CAP-1D5 cells in PEM medium; TCID₅₀ value is the virus titer in number of viruses/ml, which is necessary to infect 50% of the host cells.

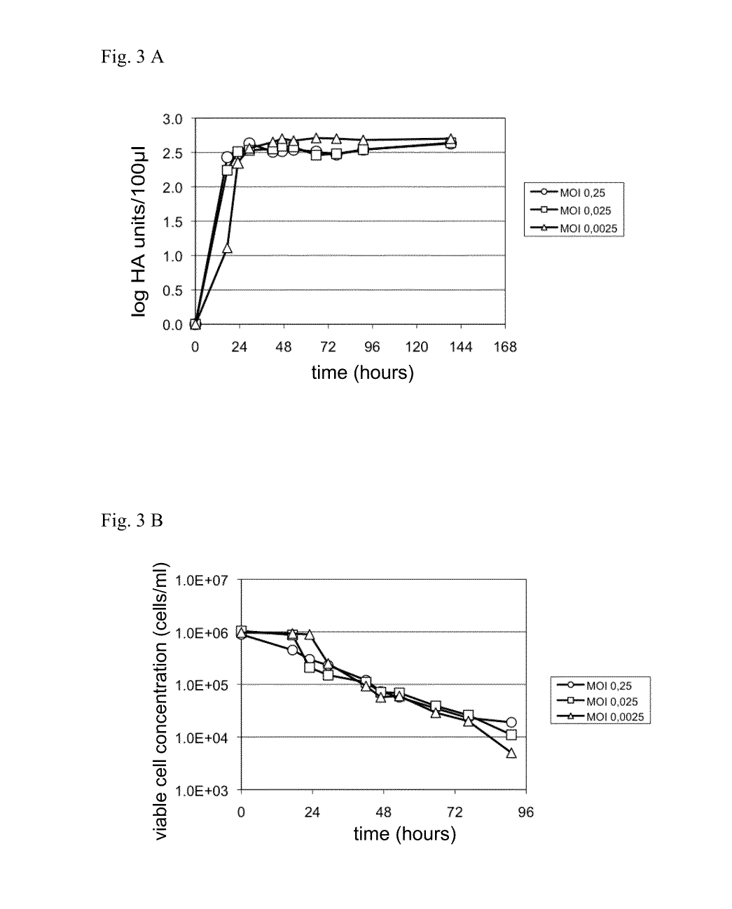

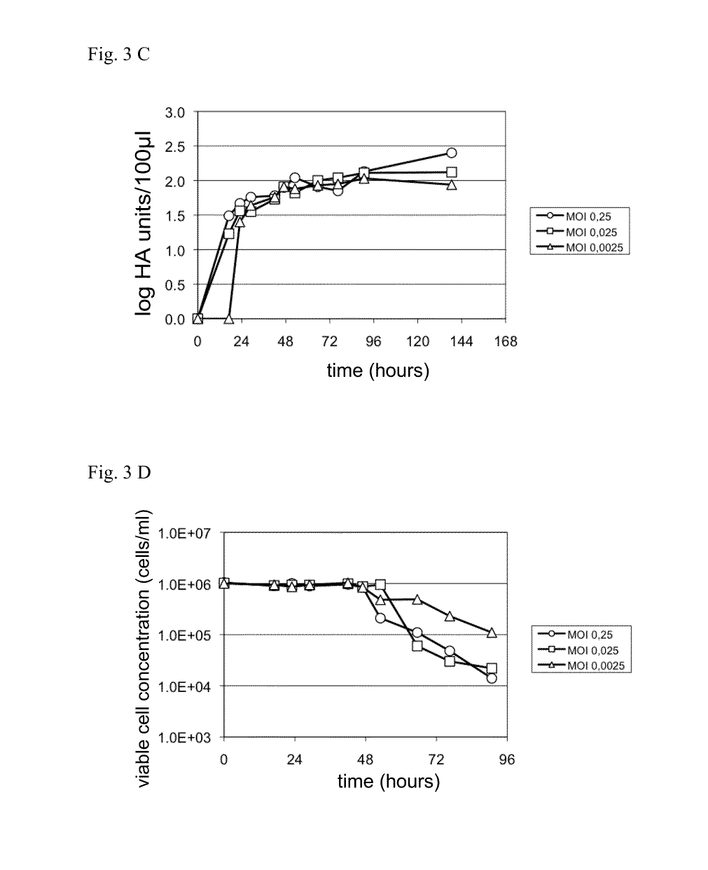

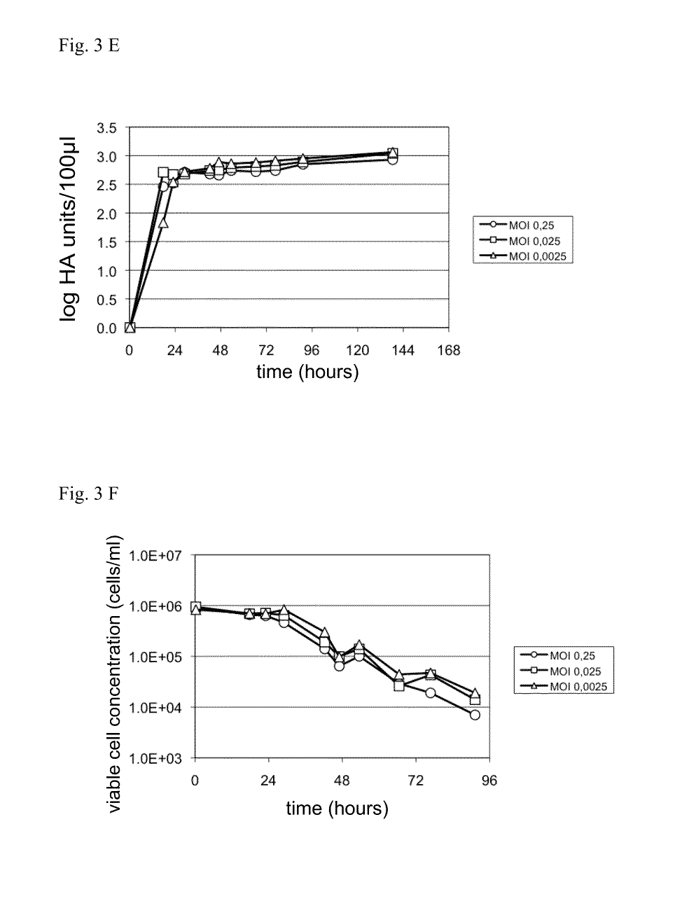

FIG. 3 A to F shows schematically the course of the amount of virus particles specified as log HA (hemagglutinin) units/100 μl and the viable cell concentration in the culture of permanent amniocyte cells CAP-1D5 in 293SFMII-(A, B) and in PEM medium (C, D), and the permanent canine kidney cells MDCK.SUS2 in SMIF8 medium (E, F) after infection of the cells by the influenza virus strain A/PR/8/34 when using different amounts of virus indicated as MOI (multiplicity of infection) values: MOI: 0.0025 (Δ) MOI: 0.025 (□), MOI: 0.25 (∘). MOI (multiplicity of infection) represents the ratio of the number of infectious particles to the target cells.

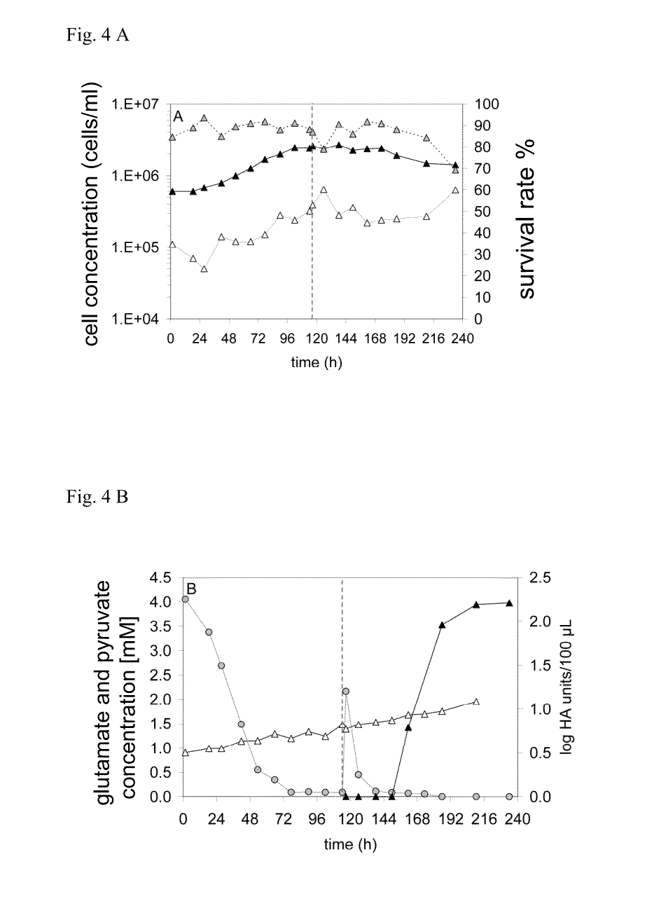

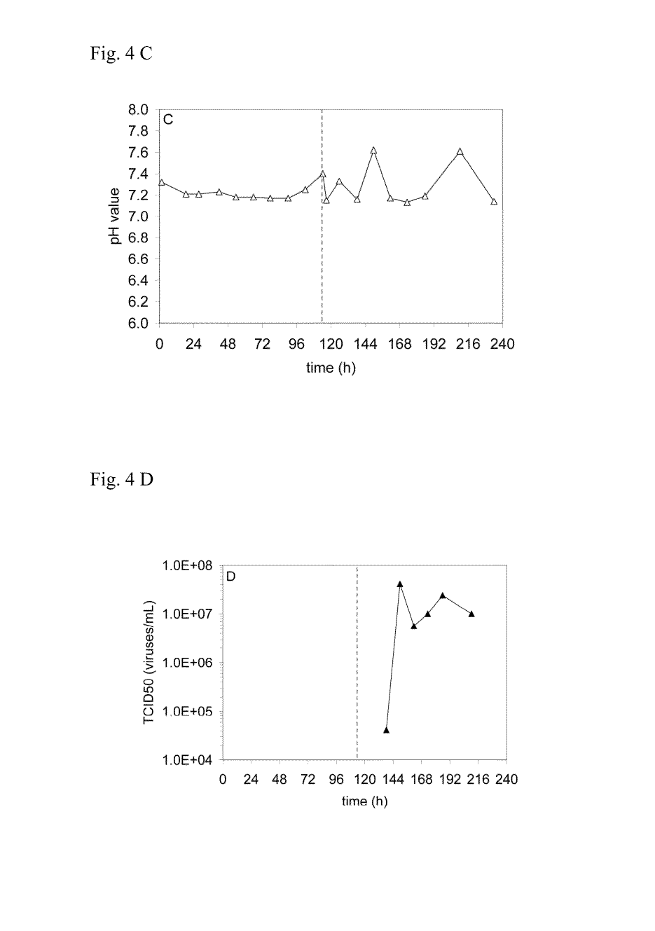

FIG. 4 A to D shows schematically the course of different parameters in the cultivation of the permanent amniocyte cell line CAP-1D5 in PEM medium in 1 L bioreactor, wherein the infection takes place after 114 h with a virus amount indicated as MOI of 0.025 with the influenza virus A/PR/8/34 (adapted). FIG. 4A shows the schematic course of the viable cell concentration (▴), dead cell concentration (Δ) and the survival rate of the cells ( [symbol] ) FIG. 4B schematically shows the quantity of virus particles, given as log HA (hemagglutinin) units/100 μl (▴), glutamate (Δ) and pyruvate ( [symbol] ) concentration in the medium. FIG. 4 C shows schematically the course of the pH value (Δ) and FIG. 4 D shows the course of the infectivity (in TCID₅₀/ml). The TCID₅₀ value indicates the virus titer in number of viruses/ml again, which is necessary to infect 50% of the host cells.

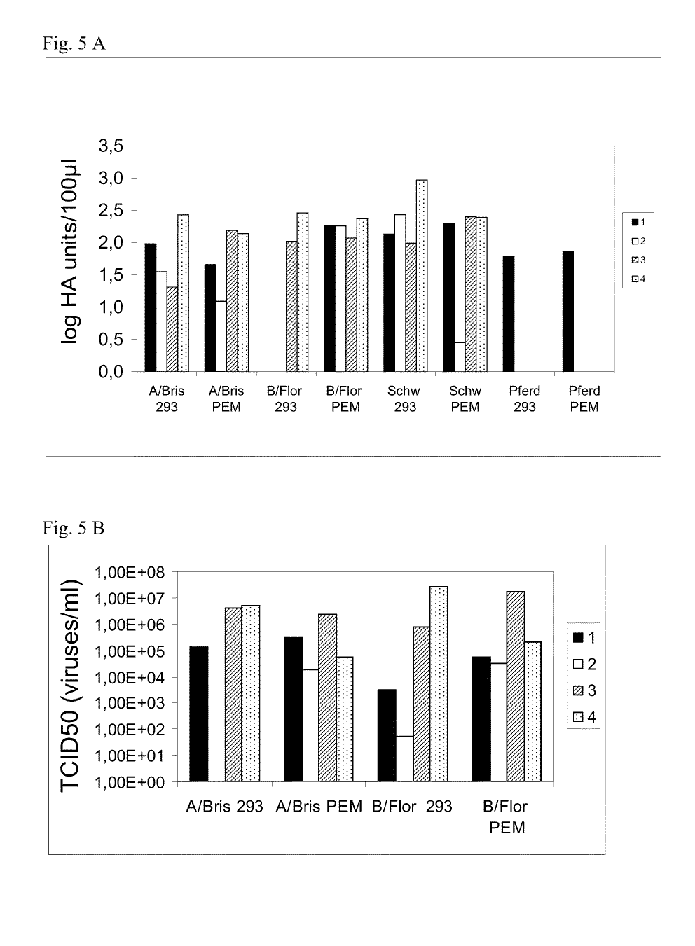

FIGS. 5 A and B show bar graphs, representing the virus titers measured as log HA units/100 μl (A) or TCID₅₀ value (B) over 4 passages of influenza strains A/Brisbane/59/2007, B/Florida/4/2006, swine influenza (A/Swine (H1N2) Bakum/1832/00) and equine influenza (A/Equine, A/Newmarket/1/93 (H3N8)) on CAP-1D5 cells in 293SFMII and PEM medium. Abbreviations: A/Bris 293: influenza strain A/Brisbane/59/2007 on CAP-1D5 cells in 293SFMII medium; A/Bris PEM: influenza strain A/Brisbane/59/2007 on CAP-1D5 cells PEM medium; B/Flor 293: influenza strain B/Florida/4/2006 on CAP-1D5 cells in 293SFMII medium; B/Flor PEM: influenza strain B/Florida/4/2006 on CAP-1D5 cells in PEM medium; Schw 293: influenza strain A/Swine (H1N2) Bakum/1832/00 on CAP-1D5 cells in 293SFMII medium; Schw PEM: influenza strain A/Swine (H1N2) Bakum/1832 00 on CAP-1D5 cells in PEM medium; horse 293: influenza strain A/Equine, A/Newmarket/1/93 (H3N8) on CAP-1D5 cells in 293SFMII medium; horse PEM: influenza strain A/Equine, A/Newmarket/1/93 (H3N8) on CAP-1D5 cells in PEM medium; TCID₅₀ value is the virus titer in number of viruses/ml, which is necessary to infect 50% of the host cells.

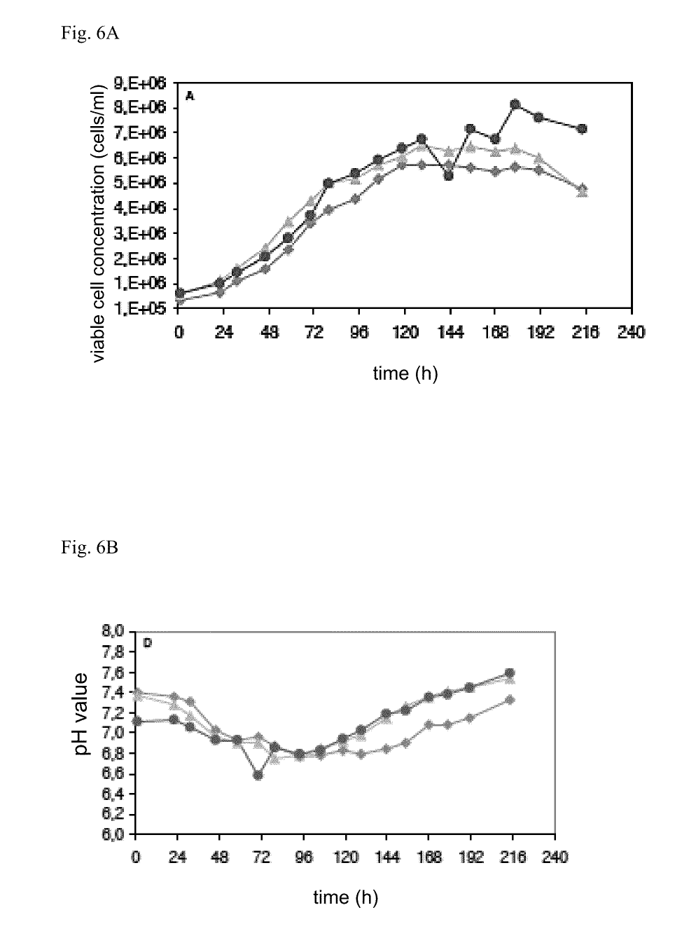

FIGS. 6 A and B schematically shows the course of the viable cell concentration and the pH value in the cultivation of the permanent amniocyte cell line CAP-1D5 in 100 ml of PEM medium in shake flasks, wherein the initial cell concentration is 5×10⁵ cells/ml and the medium additionally contains 4 mM pyruvate (♦), or the initial cell concentration is 8×10⁵ cells/ml, and the medium additionally contains 4 mM pyruvate (▴), or the start cell concentration is 8×10⁵ cells/ml and the medium additionally contains 10 mM pyruvate plus further amino acids ().

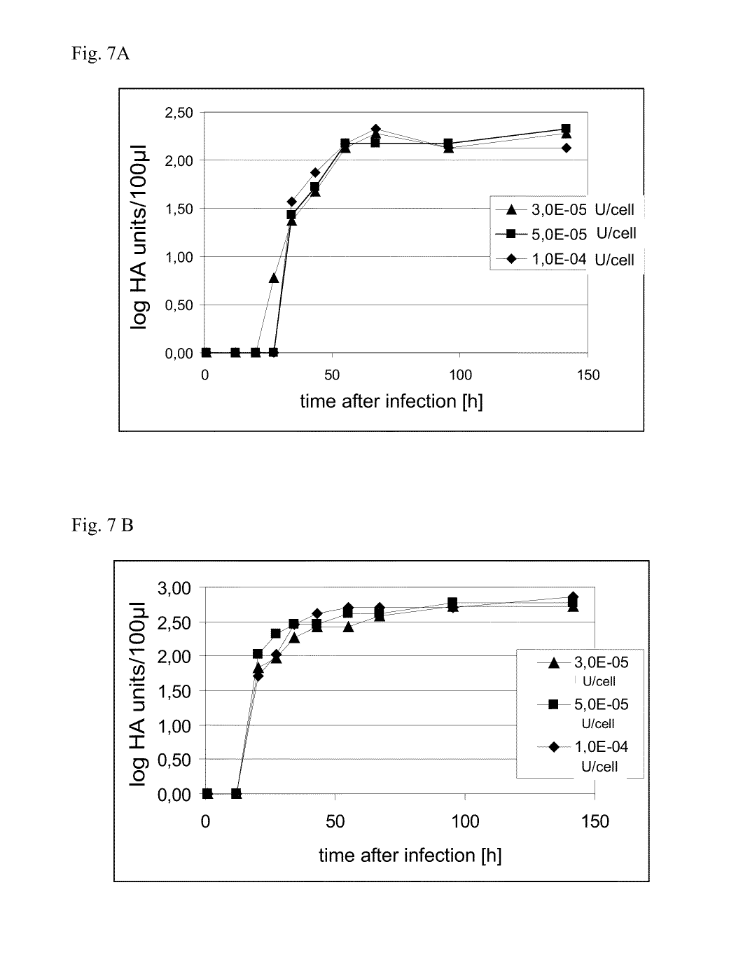

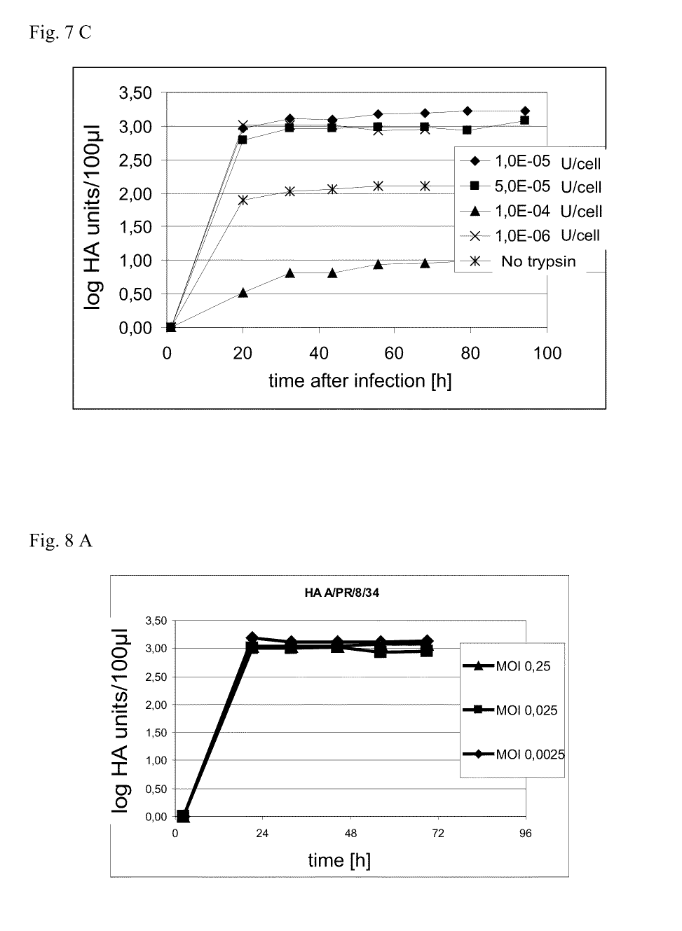

FIG. 7 A to C schematically shows the course of the virus titers measured in log HA units/100 μl culture, wherein the CAP-1D5 cells were infected with the adapted influenza strain A/PR/8/34. Before the infection, either no change of medium (A), a 1:2 dilution with PEM medium (B) or a complete medium change was performed. FIG. 7 A shows the schematic course of the virus titer of CAP-1D5 cell cultures without changing the medium, wherein different trypsin concentrations of 1×10⁻⁴ U/cell (♦), 3×10⁻⁵ U/cell (▴) and 5×10⁻⁵ U/cell (▪) were used for the infection. FIG. 7 B shows the schematic course of the virus titer of CAP-1D5 cell cultures with a 1:2 dilution with PEM medium wherein different trypsin concentrations of 1×10⁻⁴ U/cell (♦), 3×10⁻⁵ U/cell (▴), and 5×10⁻⁵ U/cell (▪) were used in the infection. FIG. 7 C shows the schematic course of the virus titer of CAP-1D5 cell cultures with complete medium change, wherein either no trypsin ( [symbol] ) or different trypsin concentrations of 1×10⁻⁴ U/cell (▴),1×10⁻⁵ U/cell (▴), 5×10⁻⁵ U/cell (▪) and 1×10⁻⁶ U/cell (x) were used for the infection.

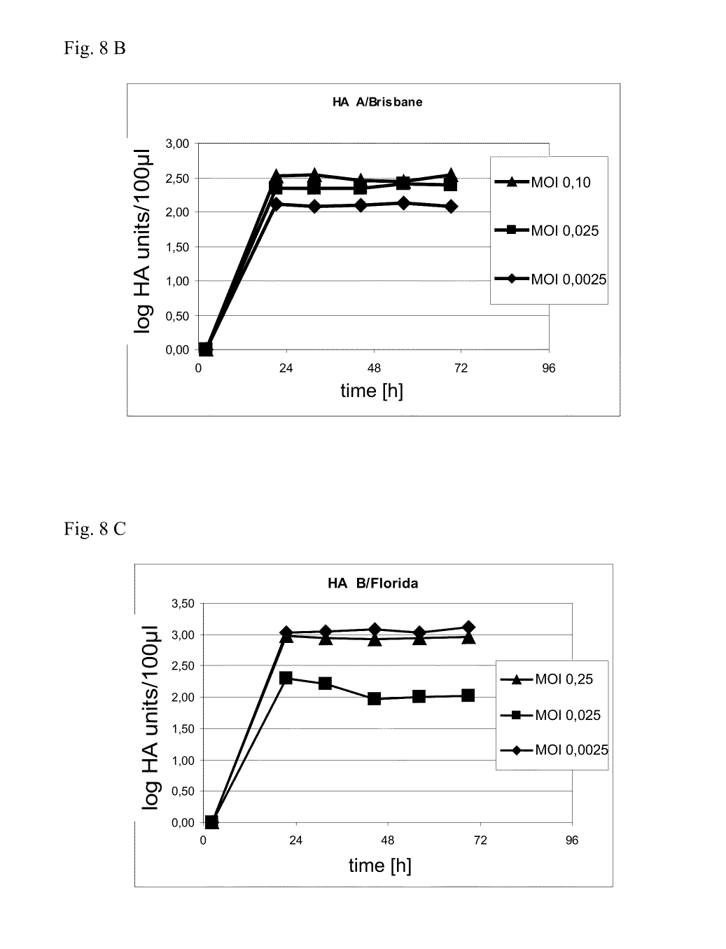

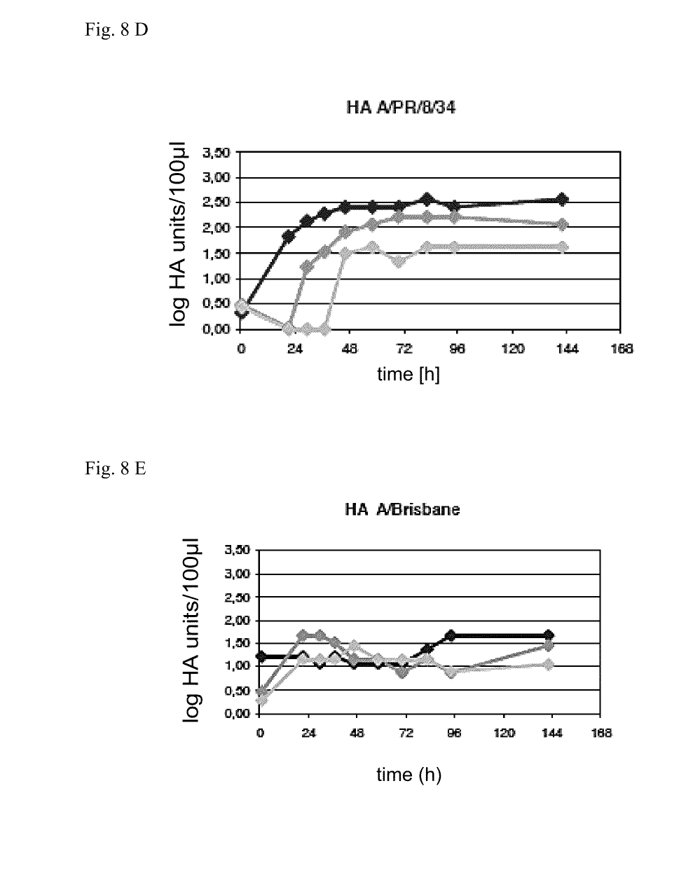

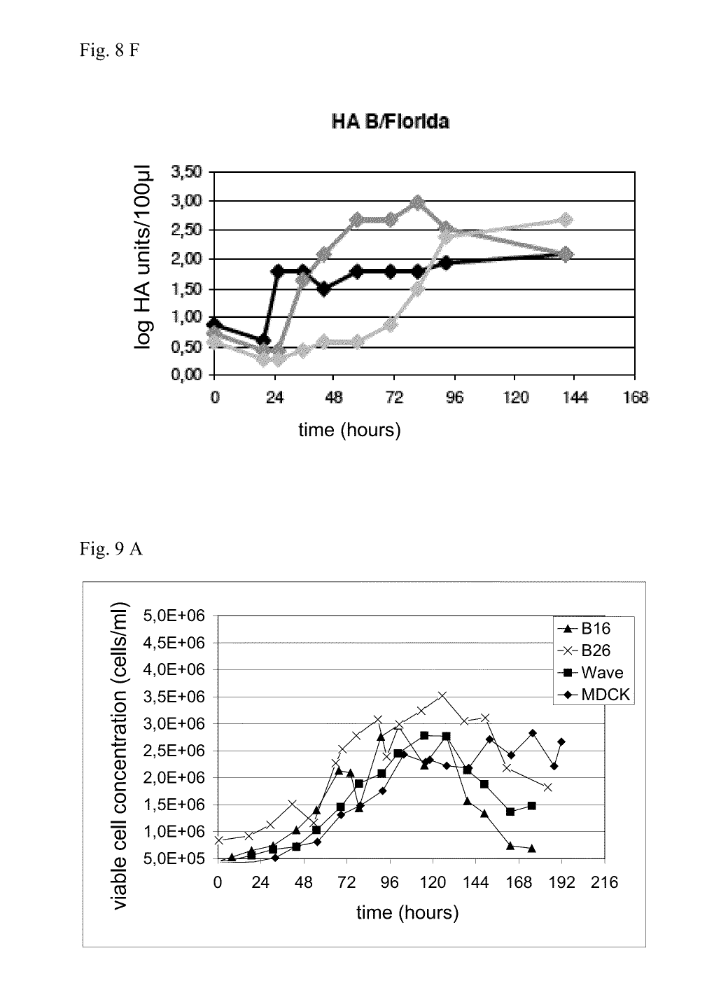

FIG. 8 A to F shows schematically the course of the virus titer in CAP-1D5 cell cultures which were infected with the influenza viruses A/PR/8/34, A/Brisbane/59/2007 or B/Florida/4/200, wherein before the infection a medium change was performed (A to C) or not (D to F). The infection with the influenza strain A/PR/8/34 and B/Florida/4/2006 was respectively done with amounts of virus indicated as MOI of 0.25, 0.025 and 0.0025. Infection with the influenza strain A/Brisbane/59/2007 respectively took place at the MOI values of 0.1, 0.025 and 0.0025. MOI (multiplicity of infection) represents the ratio of the number of infectious particles to the target cells.

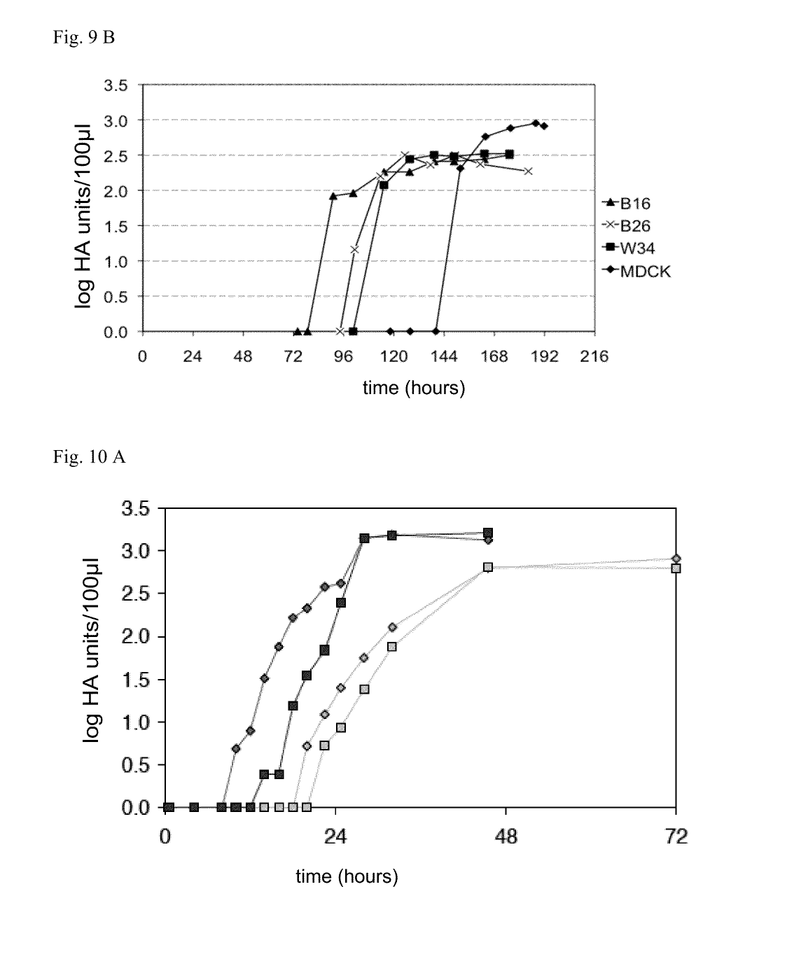

FIGS. 9 A and B shows schematically the course of viable cell concentration and the virus titer of CAP-1D5-cell cultures (B16, B26, and Wave) and a canine kidney-MDCK. SUS2 culture (MDCK), which were infected with adapted A/PR/8/34 influenza virus and cultivated in 1 L scale in STR (Sartorius) (B 16, B26, and MDCK) or Wave Bioreactors (Wave Biotech AG) (Wave). Prior to the infection, in case of the B26 cultures and Wave a medium change took place.

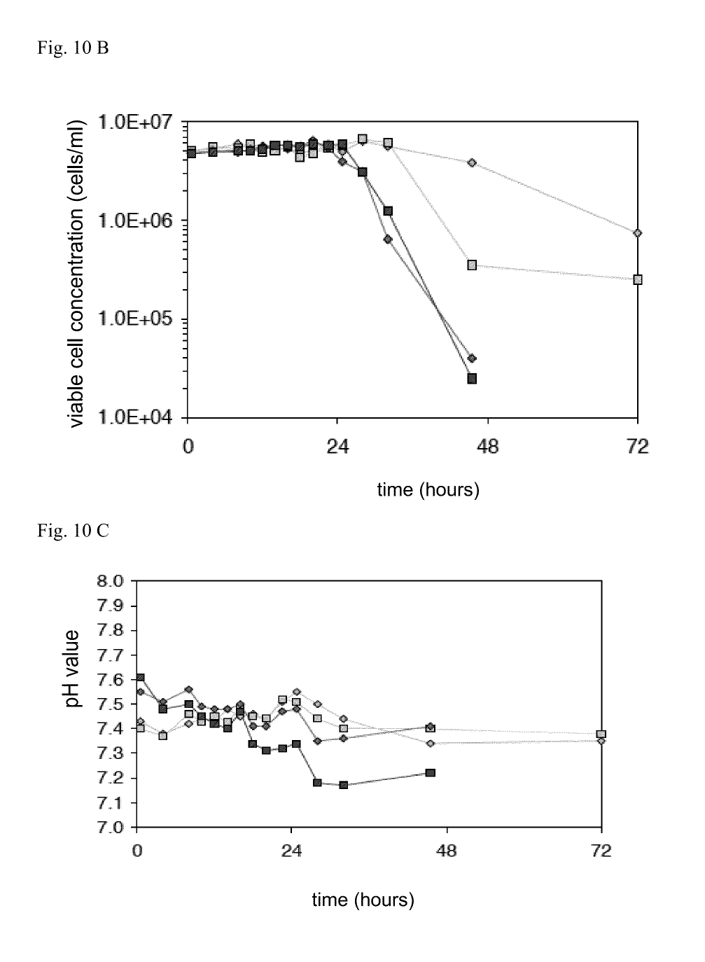

FIG. 10 A to C shows schematically the course of the virus titer measured in log HA units/100 μl, the viable cell concentration and the pH value of CAP-1D5 cell cultures, which were infected with an adapted influenza virus A/PR/8/34 and cultivated in PEM 100 ml medium in shake flasks. Prior to infection there was either a 1:1 medium change (bright symbols) with 293SFMII medium (□) or PEM medium (⋄) or a complete change of medium (dark symbols) with 293SFMII medium (▪) or PEM medium (♦).

The term “influenza virus” as used herein refers to members of the orthomyxoviruses, which can infect humans and animals. They are classified as influenza virus types A, B and C. Influenza A and B viruses are summarized in a genus. Influenza C viruses are distinguished due to their seven genome segments. The influenza A and B viruses have eight genome segments. In addition, influenza A and B viruses each encode a hemagglutinin (HA) and a neuraminidase (NA); In contrast, the influenza C viruses encode a surface protein, which combines the two properties the hemagglutinin-esterase-fusion protein (HEF). The Influenza A viruses are further divided into sub-types, based on the sequence of hemaglutinin (H1-H15) and neuraminidase (N1-N9) molecules.

The term “influenza virus protein” as used herein, refers to proteins or derivatives of the influenza virus. A derivative of the influenza virus is typically a protein or a part thereof of the influenza virus, which may be used for immunization purposes. Influenza virus proteins or derivatives thereof comprise proteins of the viral envelope or parts thereof. Particularly, influenza virus proteins comprise influenza A proteins, influenza B proteins or influenza C proteins, e.g. hemagglutinin (HA), neuraminidase (NA), nucleoprotein (NP), the matrix proteins (M1) and (M2), the polymerase proteins (PB1), (PB2) and (PA) and the non-structural proteins (NS1) and (NS2) and parts thereof. Parts of the influenza virus proteins comprise one or more epitopes of the influenza A proteins, influenza B proteins or influenza C proteins. The epitopes may be CD4+ T-cell epitopes, which represent peptides containing a binding motif of class MHC class II and are represented on the surface of the antigen presenting cells, by molecules of the MHC class II, or CD8+ T-cell epitopes, which are peptides containing a binding motif of the class MHC class I and are represented on the surface of antigen-presenting cells by molecules of the MHC class-I. For example, algorithmic model, MHC binding assays, in silico antigen identification methods, and X-ray crystallographic methods allow the identification of antigens which may bind different MHC molecules.

The term “vaccine” as used herein refers to a biologically or genetically engineered antigen, comprising proteins, protein subunits, peptides, carbohydrates, lipids, nucleic acids, killed or attenuated viruses, wherein it may be herein whole virus particles or parts of virus particles, or combinations thereof. The antigen may be at least an epitope, e.g. a T-cell and/or B-cell epitope. Said antigen is detected by immunological receptors, such as the T-cell receptor or B-cell receptor. The vaccine is used after application for a specific activation of the immune system regarding a particular virus. Thereby, the reaction of the immune system is used to cause an immune response in the presence of viruses and their specific antigens, respectively. This leads to the formation of antibodies and specialized T-helper cells, which can provide long-lasting protection against the particular disease, which may, depending on the virus, last a few years to the entire life. Vaccines comprise live or inactivated vaccines. The live vaccine contains for example attenuated viruses still capable of reproducing viruses that cannot cause the disease. In case of an inactivated vaccine, these viruses are killed or it contains only fragments of the virus (antigens). The inactivation (killing) of the virus, for example, occurs by chemical substances, such as formaldehyde, beta-propiolactone and psoralene. The viral envelope remains maintained. There are also toxoid vaccines containing only the biologically inactive part (toxoid) of the toxin of a virus (e.g. the tetanus toxoid), which are also included among the dead vaccines. In particular, the inactivated vaccine may be a split vaccine, consisting of fragments of the virus envelope proteins. The destruction or splitting of the viral envelope can occur for example with detergents or strong organic solvents. The viruses can be inactivated and killed in addition with chemical agents, respectively. Further the subunit vaccines are part of the dead vaccines; they consist of specific components of the virus, for example hemaglutinin and neuraminidase proteins.

The term “influenza virus-based vaccine” as used herein, refers to all proteins, peptides or parts thereof as well as nucleic acids encoding these proteins, peptides or parts thereof of the influenza virus, as well as influenza virus particles themselves, recombinant influenza virus proteins, including influenza envelope proteins, sub-viral particles, virus-like particles (VLP), VLP-complexes, and/or parts thereof, which may be used for immunization purposes against influenza.

The term “adjuvant” as used herein refers to substances which can modulate the immunogenicity of an antigen. Adjuvants are, for example, mineral salts, squalene mixtures, muramyl peptides, saponine derivatives, mycobacterial cell wall preparations, certain emulsions, monophosphoryl lipid A, mycolic acid derivatives, nonionic block copolymer surfactants, Quil A, subunit of the cholera toxin B, polyphosphazenes and derivatives thereof, immune-stimulating complexes, cytokine adjutants, MF59 adjuvant, lipid adjuvants, mucosal adjutants, certain bacterial exotoxins, specific oligonucleotides, and PLG.

The term “amniocyte” as used herein, refers to all cells that are present in the amniotic fluid and may be obtained by amniocentesis. They derive either from the amnion or fetal tissue, which is in contact with the amniotic fluid. Three major classes of amniocytes were described, which are differentiated on the basis of morphological criteria: Fibroblast-like cells (F-cells), epithelioid cells (E-cells) and amniotic fluid cells (amniotic fluid cells, AF cells) (Hohn et al, Pediat. Res 8:746-754, 1974). AF cells are the predominant cell type.

The term “permanent cell lines” as used herein refers to cells that are genetically modified such that they may permanently grow in a cell culture under appropriate culture conditions. Such cells are also referred to as immortalized cells.

The term “primary cells” as used herein refers to cells which have been obtained by direct extraction from an organism, or a tissue, and taken into the culture. Primary cells have only a very limited life span.

The term “transfection” as used herein, refers to any procedure which is suitable for the introduction of said nucleic acid(s) into the cells. Examples include the conventional calcium phosphate method, electroporation, liposomal systems of all types and combinations of these methods.

The term “CAP” as used herein, refers to permanent human amniocyte cells lines, which were generated by immortalization of primary human amniocytes with adenoviral E1A and E1B gene functions.

The term “CAP-T” as used herein, refers to CAP cells which were in addition transfected in a stabile manner with a nucleic acid molecule containing the sequence of the SV40 large T-antigen.

An object of the present invention relates to a method for the production of an influenza virus based vaccine, comprising the following steps:

(i) contacting an influenza virus with a permanent human cell,
(ii) culturing the permanent human cell,
(iii) allowing the expression of the influenza virus, and
(iv) isolating the influenza virus from the medium.

In the method according to the present invention, permanent human cells are cultured under conditions (e.g. temperature, medium, pH) that are suitable for the growth of the cells. The conditions in terms of temperature, the medium, the pH value and other growth parameters, are known by those skilled in the art, or may be determined by the usual methods. As the culture has reached a desired growth density, the influenza viruses are added for infection of the cells. The virus may take several days for propagation within the cells. During this reproduction process, a large part of the cells will die and the viruses are released into the medium. The virus-containing solution is separated from the cell debris, for example by centrifugation. The virus may then be separated from the medium solution by means of e.g. a chromatography column, and the volume may be reduced. The viruses may then be inactivated, for example by a chemical process. This may be followed by a viral splitting. After further purification and concentration steps, the antigen concentrate of a virus strain is obtained.

In a preferred embodiment, the influenza virus strains A/PR/8/34, A/Uruguay/716/2007, A/Brisbane/59/2007, B/Florida/4/2006, swine influenza (A/Swine (H1N2) Bakum/1832/00) or equine influenza (A/Equine, A/Newmarket/1/93 (H3N8)) are used for infection of the permanent human cells.

In a further preferred embodiment, the influenza viruses used for the infection of the permanent human cells will be previously adapted to the cells; preferably, these are the above-listed influenza viruses. Preferably, such an adaptation is over 4 passages. Preferably, the adaptation of influenza viruses occurs in 293SFMII medium or PEM medium.

A further object of the present invention relates to a method for the production of an influenza virus based vaccine comprising the following steps:

(i) contacting a nucleic acid molecule, encoding an influenza virus protein with a permanent human cell,
(ii) culturing the permanent human cell,
(iii) allowing the replication of the nucleic acid molecule encoding an influenza virus protein and/or expression of the influenza protein, and
(iv) isolating the nucleic acid molecule encoding an influenza protein and/or the influenza virus protein from the medium.

In a preferred embodiment, the permanent human cells used in the method according to the present invention are permanent human amniocyte cells.

In a preferred embodiment of the present invention, the permanent human cells are cultivated in shake flasks or bioreactors, preferably STR or Wave Bioreactors. The permanent human cells may be cultured in various media, but preferably in 293SFMII or PEM medium. Further, pyruvate, glutamine, glucose, and other amino acids may be added to the medium. Preferably, the medium contains 4 mM or 10 mM pyruvate and other amino acids.

In a further preferred embodiment of the present invention, the initial cell concentration of permanent human cells, when cultivated in shake flasks, is 5×10⁵ cells/ml, more preferably 8×10⁵ cells/ml.

In a further preferred embodiment of the present invention, the pH value of the cell culture is in the range of 7.1 to 7.8, more preferably in the range of 7.3 to 7.5, even more preferably in the range of 7.3 to 7.5.

In a further preferred embodiment of the present invention, a complete change of medium, or a 1:2 dilution of the medium, is carried out prior to the infection of permanent human cells with influenza virus.

In a preferred embodiment of the present invention the trypsin concentrations of 1×10⁻⁴ U/cell, 1×10⁻⁵ U/cell, 3×10⁻⁵ U/cell, 5×10⁻⁵ U/cell or 1×10⁻⁶ U/cell will be used to infect the human permanent cells with influenza virus. If there is no medium change prior to the infection of human permanent cells, a trypsin concentration of 1×10⁻⁴ U/cell is preferably used for the infection of the cells with influenza virus. If a 1:2 medium dilution is performed prior to the infection of the human permanent cells, a trypsin concentration of 5×10⁻⁵ U/cell is preferably used for the infection of the cells with the influenza virus. If a complete change of medium is performed prior to infection of the human permanent cells, a trypsin concentration of 5×10⁻⁶ U/cell is preferably used for the infection of the cells with the influenza virus.

In a preferred embodiment of the present invention, a virus amount which is specified as MOI (multiplicity of infection) value in the range of 0.001 to 0.3 is used for the infection of permanent human cells. In a preferred embodiment of the present invention a virus amount specified as MOI (multiplicity of infection) value of 0.25, 0.1, 0.06, 0.025 or 0.0025 is used for the infection of the permanent human cells. Preferably, when the permanent human cells are infected with the influenza virus A/PR/8/34 without performing a medium change prior to the infection, a virus amount indicated as MOI value of 0.25 is used in the infection of permanent human cells with influenza virus; when the permanent human cells are infected with the influenza virus A/Brisbane/59/2007 without performing a medium change prior to the infection, a virus amount indicated as MOI value of 0.1 is used in the infection of permanent human cells with influenza virus. Preferably, when the permanent human cells are infected with the influenza virus A/PR/8/34 with performing a medium change, a virus amount indicated as MOI value of 0.1 or 0.25 is used in the infection of permanent human cells with influenza virus; when the permanent human cells are infected with the influenza virus A/Brisbane/59/2007 with performing a medium change, a virus amount indicated as MOI value of 0.06 or 0.25 is used in the infection of permanent human cells with influenza virus; and when the permanent human cells are infected with the influenza virus B/Florida/4/2006 with performing a medium change, a virus amount indicated as MOI value of 0.01, 0.025 or 0.0025 is used in the infection of permanent human cells with influenza virus.

In a preferred embodiment, the cell concentration at the time of infection in case of cultivation in shake flasks is in a range from 1×10⁶ to 6×10⁶ cells/ml. Preferably, the cell concentration is at the time of infection 2.3×10⁶ cells/ml, 4.5×10⁶ cells/ml or 5×10⁶ cells/ml. In a preferred embodiment of the present invention, the cell concentration at the time of infection is 4.5×10⁶ cells/ml, and no medium change is performed prior to the infection. In a further preferred embodiment of the present invention, the cell concentration at the time of infection is 2.3×10⁶ cells/ml, and prior to the infection a dilution of 1:2 with fresh PEM medium is performed. In a further preferred embodiment of the present invention, the cell concentration at the time of infection is 5×10⁶ cells/ml, and a complete change of medium is performed prior to the infection.

In a particularly preferred embodiment of the present invention, the permanent human cells are cultured in a 1 liter bioreactor STR (Sartorius) in PEM medium with 4 mM glutamine and 4 mM pyruvate, wherein the initial cell concentration is 5×10⁵ cells/ml, and wherein at a cell concentration of 2.1×10⁶ cells/ml with influenza virus in a quantity indicated as MOI value of 0.025 is infected and no medium change is performed prior to infection. Preferably, the infection is carried out in the presence of trypsin in a final concentration of 3×10⁻⁵ U/ml.

In a particularly preferred embodiment of the present invention, the permanent human cells are cultivated in a 1 liter bioreactor STR (Sartorius) in PEM medium, wherein the initial cell concentration is 8×10⁵ cells/ml, and wherein infection is performed with influenza virus using an amount of virus indicated as MOI value of 0.025, and wherein a medium change is performed prior to infection. Preferably, the infection is carried out in the presence of trypsin in a final concentration of 3×10⁻⁵ U/ml.

In another particularly preferred embodiment of the present invention, the permanent human cells are cultured in the 1 liter Wave bioreactor (Wave Biotech AG) in PEM medium with 4 mM glutamine, 4 mM pyruvate and 20 mM glucose in PEM medium, the initial cell concentration is 5×10⁵ cells/ml, and wherein the cell concentration prior to infection is 2.1×10⁶ cells/ml and it is infected with influenza virus using an amount of virus given as MOI value of 0.025 and no medium change is performed prior to infection. Preferably, the infection is carried out in the presence of trypsin in a final concentration of 3×10⁻⁵ U/ml.

In a preferred embodiment of the present invention, the permanent human cells are cultivated in PEM medium with 4 mM glutamine and 4 mM pyruvate in shake flasks, wherein a medium change is performed prior to infection of the cells with influenza virus, using an amount of virus indicated as MOI value of 0.025 in the presence of a trypsin concentration of 1×10⁻⁶ U/cell.

In a preferred embodiment of the present invention, the permanent human cells are cultured in PEM medium with 4 mM glutamine and 4 mM pyruvate in shake flasks, wherein a 1:1 medium change is performed prior to infection of the cells with influenza virus, using an amount of virus indicated as MOI value of 0.025 in the presence of a trypsin concentration of 1×10⁻⁵ U/cell. In the production of influenza proteins and nucleic acid molecules encoding an influenza protein, the cultured human cells with nucleic acid molecules encoding an influenza protein will be transfected, and subsequently the influenza virus protein or the nucleic acid molecules encoding an influenza protein will be isolated and purified, using known methods.

In a further preferred embodiment, the human cells are in or between the mid-exponential growth phase and the stationary growth phase in the method according to the present invention at the time of infection with a virus particle, or at the time of transfection with a nucleic acid molecule encoding an influenza virus protein or part thereof. A typical growth curve in which the cell concentration is mapped against time has a sigmoid curve shape. It begins with a so-called lag phase, followed by the log phase or exponential phase and the stationary phase. The middle exponential growth phase in this case corresponds to the first inflection point of a typical growth curve, wherein the inflection point is a point on the growth curve, where the shape of the curve course changes from concave to convex or from convex to concave. The stationary phase begins when the growth curve reached a plateau, and thus the number of cells remains constant.

The nucleic acids produced by the method according to the present invention which encode a protein of influenza, provided by the inventive method, may be used for nucleic acid immunization, or as the so-called DNA vaccines. In nucleic acid immunization, immunogenic antigens, i.e. antigens which elicit an immune response in humans, are inoculated. These immunogenic antigens are encoded by DNA or RNA, and are present as expression cassettes or vectors, or are integrated into viral vectors in order to induce an immune response to the gene product. DNA vaccines may be provided in different delivery systems, e.g. as DNA or RNA, in the form of linearized or circular plasmids or expression cassettes, wherein they are provided with the necessary elements for expression, such as a promoter, polyadenylation sites, origin of replication, etc. In case of administration of DNA, same is usually present in a buffer with or without adjuvant or bound to nanoparticles or in an adjuvant-containing compound or integrated in a viral or bacterial vector. DNA vaccines elicit both humoral and cell-mediated immunity. An advantage of the DNA vaccine is that the antigen is expressed in its native form, and thus leads to an improved immunization. Another advantage of the DNA vaccine is that, by contrast to weakened live vaccines, it is not infectious and may not be made virulent again. The administration of the DNA vaccine in the form of DNA or RNA, of plasmids or linear DNA fragments, which are coupled to particles, may be carried out by injection or by means of a gene gun. Here, for example, the DNA vaccine for injection, is present in a saline or buffered saline solution.

The nucleic acids produced by the method according to the present invention which encode influenza protein, influenza protein and influenza virus, may be used as a vaccine against the influenza virus type A and/or B and/or C.

The influenza virus based vaccine, produced by the method according to the present invention, comprises all proteins, peptides or parts thereof, as well as nucleic acids which encode these proteins, peptides or parts thereof, of the influenza virus, as well as influenza virus particles itself, recombinant influenza virus proteins, including influenza envelope proteins, sub-viral particles, virus-like particles (VLP), VLP-complexes, and/or parts thereof, which may be used for immunization purposes against influenza.

Preferably, the influenza proteins produced by the method according to the present invention are proteins or derivatives of influenza virus, preferably of the influenza virus strains A/PR/8/34, A/Uruguay/716/2007, A/Brisbane/59/2007, B/Florida/4/2006, swine influenza (A/Swine (H1N2) Bakum/1832/00) or equine influenza (A/Equine, A/Newmarket/1/93 (H3N8)).

The isolation and purification of the nucleic acids encoding an influenza virus protein or part thereof, produced by the method according to the present invention, is performed by means of the usual methods that are known to the person skilled in the art.

The isolation and purification of the influenza virus proteins, produced by the method according to the present invention, is performed by means of the usual methods that are known to the person skilled in the art. The purification of proteins initially depends on their origin. A distinction is made between intra- and extracellular proteins. If the proteins are located within the cell bodies, breaking the cells is necessary first, which is performed e.g. by shear forces or osmolysis. Thereafter the separation of insoluble material, such as cell membranes and cell walls, is done, e.g. by centrifugation. Centrifugation is used by default for the separation of cells, cell organelles and proteins. A more effective method in terms of the separation capacity is pulse electrophoresis. Additionally, after separation of other cell components, there is still the need to separate different sized proteins, peptides and amino acids. The separation of proteins may be done by one or two-dimensional gel electrophoresis or capillary electrophoresis. In the field of amino acids and peptides, for example, chromatographic methods, such as affinity chromatography, ion exchange chromatography (IEC), or reversed-phase chromatography (RPC) are used. The presence of lipids and the necessity of removal or deactivation of proteases is disadvantageous with regard to the purification. Proteins present in the extracellular matrix need not be extracted from the cells, but, after separation of all insolubles, they are highly diluted and usually in much smaller quantities than as intracellular proteins.

For the isolation and purification of the influenza-virus particles produced by the method according to the present invention, methods are used which are known to the skilled person. Examples for these methods are the density gradient differential or zonal centrifugation.

The permanent human cells used in the method according to the present invention are generated by immortalization of primary human cells. Primary human cells are obtained by direct extraction from the organism, or a tissue extracted from an organism and taken in culture. Preferred are such primary human cells which are well converted into permanent human cell lines by expression with cell transforming factors, in particular amniocytes, embryonic retina cells and embryonic cells of neuronal origin.

Cell-transforming factors are T-antigen of SV40 (Genbank Acc. No. J02400), E6 and E7 gene product of HPV (e.g. HPV 16, Genbank Acc. No. K02718) and E1A and E1B gene products of human adenoviruses (e.g. human adenovirus serotype-5, Genbank Acc. No. X02996). The primary cells are transfected for the immortalization by the expression of the human adenovirus E1 with the two nucleic acid sequences for the E1A and E1B gene products. In case of expression by a naturally available HPV, E6 and E7 may be expressed from a RNA transcript. The same applies to the expression of E1A and E1B of a naturally occurring adenovirus. The cell transforming factors, such as the adenoviral E1 gene function, cause the immortalization or transformation and thus the long-term cultivability of the cells.

The expression of the cell transforming factors may be carried out under the control of a homologous promoter, and transcriptional termination elements, e.g. the natural E1A promoter and the natural E1A polyadenylation site for the expression of the adenoviral E1A gene function. This can be achieved by using the nucleic acid molecules used for the transfection of the respective viral genome fragments, e.g. of the adenoviral genome, which contains said gene functions, such as E1A, E1B. The expression of cell transforming factors may also fall under the control of heterologous promoters, not naturally with the used encoding region occurring promoters or transcriptional termination elements. For example, CMV (cytomegalovirus) promoter (Makrides, 9-26 in: Makrides (Eds.), Gene Transfer and Expression in Mammalian Cells, Elsevier, Amsterdam, 2003), EF-1 a promoter (Kim et al, Gene 91st:217-223, 1990), CAG promoter (a hybrid promoter of the immediate early enhancer of the human cytomegalovirus, and a modified chicken β-actin promoter with first intron) (Niwa et al., Gene 108:193-199, 1991), human or murine pgk (Phosphoglycerate kinase) promoter (Adra et al, Gene 60:65-74, 1987.), RSV (Rous sarcoma virus) promoter (Makrides, 9-26 in: Makrides (ed.), Gene Transfer and Expression in Mammalian Cells, Elsevier, Amsterdam, 2003), or SV40 (simian virus 40) promoter (Makrides, 9-26 in: Makrides (ed.), Gene Transfer and Expression in Mammalian Cells, Elsevier, Amsterdam, 2003) may serve as heterologous promoters. For example, the polyadenylation sequences of the SV40 Large T antigen (Genbank Acc. No. J02400), or of the human G-CSF (granulocyte colony-stimulating factor, granulocyte colony stimulating factor) gene (Mizushima and Nagata, Nucl. Acids Res 18:5322, 1990) may serve as polyadenylation sites.

By transfection of primary human cells with the nucleic acid molecule, comprising the nucleic acid sequences coding for the E1A and E1B, the cells are immortalized. The nucleic acid molecule used for immortalization of primary human cells comprises E1A and E1B-nucleic acid sequences, which are preferably derived from human adenoviruses, in particular of human adenovirus serotype-5. In a preferred embodiment, the nucleic acid molecule used for immortalization comprises the nucleic acid sequence encoding the adenoviral pIX gene function, besides the E1A and E1B-coding nucleic acid sequences. The pIX polypeptide is a viral structural protein, which acts as a transcriptional activator in several viral and cellular promoters, such as the thymidine kinase and the beta-globin promoter. The transcription-activating effect of the pIX polypeptide expressed additionally in the cell can result in an increase in the expression levels of the recombinant polypeptide in the production of cell lines according to the invention, if the coding sequence of the recombinant polypeptide is under control of one of the abovementioned promoters. An exemplary sequence is given in Genbank Acc. No. X02996. In particular, the nucleic acid molecules comprise the nucleotides 1 to 4344, 505 to 3522 or the nucleotides 505 to 4079 of the human adenovirus serotype-5.

In a preferred embodiment, the nucleic acid molecule comprises for the immortalization of primary cells, in particular of the amniocytes, the adenovirus serotype-5 nucleotide sequence from nucleotide 505 to nucleotide 4079. In a further particularly preferred embodiment, the nucleic acid molecule comprises for the immortalization of primary cells, in particular of the amniocytes, adenovirus serotype 5 nucleotide sequence from nucleotide 505 to nucleotide 3522. In another particularly preferred embodiment, the nucleic acid molecule comprises for the immortalization of primary cells, in particular of the amniocytes, adenovirus serotype 5 nucleotide sequence from nucleotide 1 to nucleotide 4344, corresponding to the adenoviral DNA in HEK 293 cells (Louis et al, Virology 233rd: 423-429, 1997). Further, the immortalized human cell may express a viral replication factor. This replication factor may bind to the origin of replication (ori, “origin of replication”) of a nucleic acid molecule introduced by transfection and thereby initiating the replication of the episomal nucleic acid molecule. The episomal replication of nucleic acid molecules, in particular plasmid DNA, into cells causes a strong increase in the copy number of the transferred nucleic acid molecules, and thereby an increase in the expression of a recombinant polypeptide encoded on this molecule, as well as its maintenance over many cell divisions. Such a viral replication factor is e.g. the T antigen of Simian Virus 40 (SV40), which after binding to a sequence indicated as an SV40 origin of replication (SV40 ori, origin of replication) on the nucleic acid molecule, for example the plasmid DNA, initiates its replication. The Epstein-Barr virus EBNA-1 protein (Ebstein Barr virus nuclear antigen-1) recognizes an origin of replication designated as ori-P and catalyzes the extrachromosomal replication of the ori-P bearing nucleic acid molecule. The T-antigen of simian virus 40 (SV40) activates the replication not only as a replication factor, but also has an activating effect on the transcription of some viral and cellular genes (Brady, John and Khoury, George, 1985, Molecular and Cellular Biology, vol 5, no. 6, p. 1391 to 1399).

The immortalized human cell used in the method according to the present invention is in particular for an immortalized human amniocyte cell. In a preferred embodiment, the immortalized human cell used in the method according to the present invention expresses the large T antigen of SV40 or the Epstein-Barr virus (EBV) Nuclear Antigen 1 (EBNA-1). In a particularly preferred embodiment, the immortalized human amniocyte cell used in the method according to the present invention expresses the large T antigen of SV40 or the Epstein-Barr virus (EBV) Nuclear Antigen 1 (EBNA-1). In another particularly preferred embodiment, the immortalized human cell, in particular amniocyte cell, used in the method according to the present invention expresses the large T antigen of SV40 under control of the CAG SV40, RSV or CMV promoter.

The permanent human amniocytes used in the method according to the present invention are particularly described in the patents EP 1230354 and EP 1948789 In a particularly preferred embodiment the permanent human amniocyte cell used in the method according to the present invention is CAP or CAP-T.

In the case of the CAP cells, the primary amniocytes were transfected with a plasmid containing the murine pgk promoter, Ad5 sequences nt. 505-3522, containing the entire E1 region, the 3′ splice and polyadenylation signal of SV40 and the pIX region of Ad5 (nt. 3485-4079). This plasmid has been described in detail in EP 1 948 789.

For the production of CAP-T-cells, the CAP cells were transfected with a plasmid comprising the expression cassette for T antigen of SV 40, flanked with an intron from SV40 and a polyadenylation site. In addition, the plasmid may contain the CAG promoter (hybrid promoter consisting of the CMV enhancer and the chicken β-actin promoter) (Niwa et al., Gene 108:193-199, 1991), the RSV promoter (Rous sarcoma virus promoter) (Genbank Acc Nr. DQ075935) or the CMV promoter (early promoter of the human cytomegalovirus) (SEQ ID NO: 5). In order to generate stable cell lines, the plasmid contains a blasticidin expression cassette with the ubiquitin promoter (pUB/Bsd, Invitrogen #V512-20).

Moreover, the invention provides also a method according to the present invention in which the human cell, in particular the amniocyte cell, may grow in suspension. Further, the human cell, in particular the amniocyte cell of the method according to the present invention, may be cultured in serum-free medium.

A further object of the present invention is the use of a permanent human cell, in particular an amniocyte cell, for the production of an influenza virus based vaccine.

In a preferred embodiment, the permanent human amniocyte cell used for the production of an influenza virus based vaccine is a CAP or CAP-T cell.

The influenza virus based vaccine produced by the method according to the present invention may be an influenza virus and/or influenza virus protein or a nucleic acid molecule encoding an influenza protein. The vaccine may be administered parenterally, with a syringe. A distinction is made into intradermal, subcutaneous or intramuscular injections. The intradermal injection can be performed with a vaccination gun or a lancet. Intramuscular injection may take place in the upper arm, in the thigh or buttock. Further, the vaccine may be administered orally or nasally. The vaccine may be administered for example to humans and animals.

The influenza virus based vaccine produced by the method according to the present invention may provide both by active and passive immunization, a resistance to one or more of the influenza viruses. For active immunization, the vaccine is used after the application for specific activation of the immune system of humans and animals, with respect to a particular virus. Here, the reaction of the immune system is utilized to cause an immune response in the presence of viruses or their specific antigens. This leads to the formation of antibodies and specialized T-helper cells, which then provide a long lasting protection against the particular disease, which can, depending on the virus, be from a few years or continue throughout life.

The influenza-virus based vaccine produced by the method according to the present invention may be, for example, a live or dead vaccine. The live vaccine contains for example attenuated viruses, which still are capable of reproducing viruses but cannot cause the disease. In a dead vaccine, these viruses are killed, or it contains only fragments of the virus (antigens). The inactivation (killing) of viruses is for example done by chemical substances/substance combinations, such as formaldehyde, beta-propiolactone and psoralene. The viral envelope remains maintained. There are also toxoid vaccines containing only the biologically inactive ingredient (toxoid) of the toxin of a virus (e.g. Tetanus toxoid), which are also included among the dead vaccines. In particular, a dead vaccine may be a split vaccine, consisting of fragments of the virus envelope proteins. The destruction (splitting) of the viral envelope can occur for example with detergents or strong organic solvents. The viruses may in addition also be inactivated (killed) by chemical substances. Furthermore, the dead vaccines include subunit vaccines, consisting of specific components of the virus, for example, hemagglutinin and neuraminidase proteins.

In case of a passive immunization, the influenza virus based vaccine produced by the method according to the present invention is administered to a host (e.g. a mammal), the induced antiserum is extracted and then administered to the receiver, who is infected with at least one influenza virus.

Further, for administration the vaccine is mixed with one or more additives such as stabilizers, neutralizers, carriers and preservatives. These substances include formaldehyde, thimerosal, aluminum phosphate, acetone and phenol. In addition, the vaccine may be mixed with auxiliary materials to enhance the effect of the vaccine. These so-called adjuvants should have no pharmacological effect by themselves and are particularly to serve as a solubilizer, emulsions or mixtures thereof. Adjuvants are, for example, mineral salts, squalene mixtures, muramyl peptides, saponine derivatives, mycobacterial cell wall preparations, certain emulsions, monophosphoryl lipid A, mycolic acid derivatives, nonionic block copolymer surfactants, Quil A, subunit of cholera toxin B, polyphosphazenes, and its derivatives, immune stimulatory complexes, cytokine adjuvants MF59 adjuvant lipid adjutants, mucosal adjuvants, certain bacterial exotoxins, specific oligonucleotides and PLG.

The following examples illustrate the invention and should not be construed as limiting. Unless otherwise indicated, standard molecular biological methods were used, such as described by Sambrook et al, 1989, Molecular Cloning. A Laboratory Manual 2nd Edition, Cold Spring Harbor Laboratory Press, Cold Spring Harbor, N.Y.

### EXAMPLE 1

### Cultivation Experiments with the Permanent Amniocyte Line CAP-1D5 in PEM or 293SFMII Medium

The permanent amniocyte cell line CAP-1D5 was cultured in 100 ml of serum-free 293SFMII medium (Invitrogen) or PEM medium (Invitrogen) at 37° C., 8% CO₂ and 100 rpm. As a control, the permanent canine kidney cell line MDCK.SUS2 (Madin Darby Canine Kidney, adapted to growth in suspension) (Lohr et al., Vaccine, 2010, 28 (38):6256-64) is used in 100 ml serum free SMIF8 medium in 100 ml shake flasks.

At time 0 (=start of the culture), and in each case in a period of 24 h, the viable cell concentration, the dead cell concentration, the survival rate of the cell lines were determined, as well as the pH value, the concentration of glucose, lactose, glutamine, ammonium, glutamic acid and pyruvate in the medium (biochemical multi parameter analysis system) (Lohr et al, Vaccine, 2009, 27 (37), 4975-4982; Genzel et al, Appl Microbiol Biotechnol, 2010, 88 (2):461-75).

The results are presented in FIG. 1. The viable cell concentration of the permanent amniocyte cell line CAP 1D5 in 293SFMII, as well as in the PEM medium as well as the MDCK.SUS2 cells, starting from about 2×10⁵ cells per ml of culture at the beginning of the cultivation, showed a similar course, and achieved after 168 hours, a viable cell concentration of approximately 2×10⁶ cells per ml. From 192 h on, the viable cell concentration of MDCK.SUS2 cells fell to 9×10⁵ cells per ml and remained constant up to 240 h after the beginning of the growth curve. The viable cell concentration of permanent amniocyte cell line CAP-1D5 in the 293SFMII medium fell only at 216 h, to the initial value of 2×10⁵ cells per ml of culture, however, the viable cell concentration of the CAP-1D5 cells remained stable in PEM medium up to 240 h to about 2.5×10⁶ cells per ml of culture and, after 312 h, reached the initial value of the viable cell concentration of 2×10⁵ cells.

At the start of the growth curve and up to 168 h, the survival rate of the CAP-1D5 cells in 293SFMII medium and in PEM medium as well as the MDCK.SUS2 cells was between 80 and 90%. Up to 240 h, the survival rate of the CAP-1D5 cells in PEM medium remained at 80%, then dropped, and after 312 h was still 10%. The survival rate of the CAP-1D5 cells in 293SFMII medium was already low after 168 h and it showed about 10% after 216 h. The survival rate of the MDCK.SUS2 cells decreased constantly after 168 h was steady and reached about 45% at 240 h.

The pH value of the MDCK.SUS2 cells was relatively stable over 240 h between 7.7 and 7.6. However, the pH value was (initially 7.4) in the culture of the CAP-1D5 cells in the 293SFMII medium as well as in PEM medium steadily decreased to pH 6.4 after 240 h (293SFMII medium) and 312 h (PEM medium), respectively.

The lactose concentration increased from initial concentrations of less than 5 mM in 293SFMII medium, and in PEM medium of the respective culture of CAP-1D5 cells to 30-35 mM until 240 h. In the culture of MDCK.SUS2 cells, lactose concentration increase less strongly, but reached a similar value as the CAP-1D5 cell culture at 240 h. The glucose concentration decreased by 20 to 25 mM from the beginning of the culture of the CAP-1D5 cells in the 293SFMII medium and PEM medium, as well as in the culture of MDCK.SUS2 cells to below 10 mM, wherein the greatest decline was recorded in the culture of CAP-1D5 cells in the 293SFMII medium.

The increase of the ammonium concentration in the culture of the CAP-1D5 cells in the 293SFMII medium, as well as in the PEM medium showed a very similar course (from <0.5 to 4-5 mM). By contrast, the ammonium concentration in the culture of MDCK.SUS2 cells increased significantly stronger (to 7 mM). The decrease in glutamine concentration was again very similar in the culture of the CAP-1D5 cells in the 293SFMII medium, as well as in the PEM medium, wherein the greatest reduction was noted in the PEM medium.

The highest concentration of glutamic acid was in the culture of the MDCK.SUS2 cells and this increased only slightly. The glutamic acid concentration was higher at the beginning of cultivation in the culture of the CAP-1D5 cells in the PEM medium, by contrast to those in the 293SFMII medium and increased steadily. The pyruvate concentration in the culture of MDCK.SUS2 cells decreased to zero from 144 h on. However, the pyruvate was already used up after 48 h in the cultures of CAP-1D5 cells in the PEM medium and the 293SFMII medium, respectively.

Thus, the CAP-1D5 cells showed in the PEM medium a better growth than in the 293SFMII medium. The CAP-1D5 cells in the PEM medium showed strongest glucose consumption and strongest lactose formation. The limitation of glucose from 192 h onwards could explain the decline in cell number of CAP-1D5 cells in PEM medium. By optimization of the cultivation conditions of the CAP-1D5 cells culture in the PEM medium and 293SFMII medium, respectively, a stabilization of pH value, the addition of pyruvate and glutamine, as well as glucose might be relevant.

### EXAMPLE 2

### Viral Infection with Non-Adapted Viruses

For infection experiments with non-adapted viruses the amniocyte cell line CAP-1D5 was cultivated in 55 mL serum-free 293SFMII medium (Invitrogen) and PEM medium (Invitrogen) at 37° C., 8% CO₂, 100 rpm in shake flasks. For control the canine kidney cell line MDCK.SUS2 (Madin Darby Canine Kidney, adapted to suspension) was used.

The cell densities each listed in Table 1 were each infected with the virus B/Florida/4/2006 (NIBSC, The National Institute for Biological Standards and Control) and A/PR/8/34 (H1N1) (RKI, Robert-Koch-Institute) without changing the medium, and with the addition of 5×10⁻⁶ U/mL trypsin, respectively. The amount of produced viral particles was determined by titration of the hemagglutinin (HA, hemagglutination test by standard methods) (shown in log HA units/100 μl; Table 1) (Kalbfuss et al, 2008;. Biologicals 36 (3):145-61). Also listed in Table 1 are the respective pH values of the medium at the time.

TABLE 1
Overview of the viral infection experiments, with non-
adapted viruses in infection without medium change

| | | | Viable cells/ | pH at | H |
|---|---|---|---|---|---|
| Cell | Medium | Virus | mL at 0 hpi* | HA - max | max |
| ID5 | 293SFM | B/FLORIDA | 2.70E+06 | 6.6 | 0.87 |
| ID 5 | FEM | B/FLORIDA | 2.40E+06 | 6.6 | 0 |
| ID 5 | 293SFM | A/PR/8/34 | 2.70E+06 | 6.6 | 1.19 |
| ID5 | FEM | A/PR/8/34 | 2.50E+06 | 6.6 | 0 |
| MDCKsus | SMIF8 | B/FLORIDA | 1.50E+06 | 7.5 | 0 |
| MDCKsus | SMIF8 | A/PR/8/34 | 1.60E+06 | 7.7 | 1.84 |

hpi: Hours after infection

For permanent amniocyte cell line CAP-1D5 when cultured in PEM medium, production of virus particles could neither be found when infected with the virus B/Florida/4/2006 (B/Florida), nor with the virus A/PR/8/34. The maximum HA value for A/PR/8/34 (H1N1) in 293SFM medium was below the maximum HA value reached in MDCK.SUS2. The pH at maximum HA was comparable for all infections in CAP-1D5 (pH 6.6), but was significantly lower than in infected MDCK.SUS2 cells (pH 7.7).

Thus, an infection of permanent amniocyte cell line CAP 1D5 under the tested conditions only took place with the virus B/Florida. In the case of permanent canine kidney cell line MDCK.SUS2 (Madin Darby Canine Kidney), however, an infection was detected only for the virus A/PR/8/34 (H1N1). In subsequent experiments it was to test whether an improved infection and thus higher virus yields could be achieved by previous adaptation of the viruses to CAP-1D5 cells.

### EXAMPLE 3

### Virus Adaptation to CAP-1D5 Cells in PEM and 293SFMII Medium in Shake Flasks

The virus adaptation of the influenza viruses A/PR/8/34 (H1N1) (RKI, Robert Koch Institute), A/Uruguay/716/2007 (H3N2) (NYMC X-175C, NIBSC, The National Institute for Biological Standards and Control) and B/Florida/4/2006 (NIBSC, The National Institute for Biological Standards and Control),respectively, was performed by infection of CAP-1D5 over 4 passages in shake flasks in PEM and 293SFMII medium. Before each infection the medium was changed. The virus yield during each passage was determined by titration of the hemagglutinin (log HA units/100 μl) and by the Tissue Culture Infectious Dose₅₀ Assay (TCID₅₀ viruses/ml) was quantified (Genzel and Reichl, Vaccine production—state of the art and future needs in upstream processing in Methods of Biotechnology: Animal Cell Biotechnology—Methods and Protocols, Ed R. Pörtner, Humana Press Inc., Totowa, N.J., 2007, 457-473; Kalbfuss et al, 2008; Biological 36 (3):145-61).

The results are presented in FIG. 2. The infection of the CAP-1D5 cells with influenza virus A/PR/8/34 (H1N1) and A/Uruguay/716/2007 (H3N2) resulted both in PEM, and in 293SFMII medium to markedly increased virus titers after 4 passages. The infection of the CAP-1D5 cells with the influenza virus B/Florida/4/2006 both in cultivation in 293SFMII medium and in PEM medium led to a significant increase in virus titer in the second passage. Similar increase showed titration of the HA value during infection of the CAP-1D5 cells with the influenza virus A/PR/8/34 (H1N1) or A/Uruguay/716/2007 (H3N2) in both PEM, as well as in the 293SFMII medium.

Along with an increase in the virus titer over the adaptation, with each passage the replication of the virus was faster and could ultimately be increased significantly. Generally it appears that a slight increase in virus titers in 293SFMII medium compared to PEM medium can be achieved.

### EXAMPLE 4

### MOI (Multiplicity of Infection) Dependence of the Infection of CAP-1D5 and MDCK.SUS2 Cells with Adapted Influenza A/PR/8/34

With adapted influenza virus A/PR/8/34 (H1N1, RKI, Robert Koch Institute) the MOI-dependency was now tested at 3 different values of MOI (multiplicity of infection), the number of virus particles per host cell, 0.0025, 0.025 and 0.25 was checked. The permanent amniocyte cells in CAP-1D5 were infected in 293SFMII and PEM medium, respectively, and so were the permanent canine kidney cells in MDCK.SUS2 SMIF8 medium, in shake flasks, with various MOI of the adapted influenza virus A/PR/8/34. Upon infection, a medium change was also performed that caused a near-constant pH value around pH=7.5. Over 96 h, the viable cell concentration was now determined and the amount of virus particles (log HA units/100 μl) was now determined over 144 h by titration of the hemagglutinin in hemagglutination test with the standard methods.

The results are presented in FIG. 3. The course of the viable cell concentration, as well as the course of the amount of formed virus particles in the culture of the CAP-1D5 cells in the 293SFM medium and the culture of the MDCK.SUS2 cells in the SMIF8 medium showed no dependence on the MOI values. The virus titer increased to about 2.5 log HA-units/100 μl in the two cultures. The culture of the CAP-1D5 cells in PEM medium showed for all three MOI values lower amounts (approximately 2.0 log HA units/100 μl) of virus particles. Correspondingly, the viable cell concentration of the culture of the CAP-1D5 cells in PEM medium over 48 h remained constant at 1×10⁶ cells/ml, and then dropped to less than 1×10⁴ cells/ml. By contrast, the viable cell concentration dropped constantly in the CAP-1D5-cell cultures in the 293SFM medium and MDCK.SUS2 cell culture from 1×10⁶ cells/ml to less than 1×10⁴ cells/ml after 96 h. In all cultures, a slight delay of the virus replication was detectable at an MOI of 0.0025 proven by a time-delayed increase of the log HA units, as compared to the higher MOI values.

### EXAMPLE 5

### Cultivation in 1 L Scale with Infection (Adapted A/PR/8/34)

The CAP-1D5 cells were cultivated in a 1 liter bioreactor in PEM medium, with 4 mM glutamine and 4 mM pyruvate at 85 rpm, pH=7.2, and an oxygen partial pressure pO₂ of 40% of pure oxygen. The initial cell concentration was 5×10⁵ cells/mL.

After 114 h of growth, and a cell concentration of 2.4×10⁶ cells/ml, the CAP-1D5 cells were infected with influenza virus A/PR/8/34 (adapted: in PEM, 4th passage, 1.78×10⁷ viruses/mL). No medium change was performed, but 80 ml PEM medium were added, and also glutamine and pyruvate in a final concentration of 2 mM. The MOI value was 0.025, and trypsin in a final concentration of 1×10⁻⁵ U/mL was added. Over 240 hours and in periods of 24 hours, the viable cell concentration, and the dead concentration, the survival rate of the cell lines was determined, as well as the pH value, the concentration of glucose, lactose, glutamine, ammonium, glutamic acid and pyruvate in the medium. Further, from the time of infection (114 h),the log HA-units/100 μl and TCID₅₀ values were detected (Genzel and Reichl, Vaccine production determined—state of the art and future needs in upstream processing in Biotechnology: Animal Cell Biotechnology—Methods and Protocols, Eds., R. Partner, Humana Press Inc., Totowa, N.J., 2007, 457-473; Kalbfuss et al, 2008; Biologicals 36 (3):145-61).

The results are presented in FIG. 4. The viable cell concentration of CAP-1D5 cells initially increased up to the infection with the influenza virus A/PR/8/34 from 6×10⁵ cells/ml to 2.4×10⁶ cells/ml and slightly decreased after infection. The survival rate of the CAP-1D5 cells over the entire period of time was between 80 and 90%, and after 240 h was down to 70%. The pyruvate concentration in the culture decreased within 72 h to zero, the amount of pyruvate added in the infection with influenza virus was also used up within 10 h. The glutamate concentration increased steadily over the entire time from about 1 mM to about 1.8 mM. With a few variations, the pH value of the culture was over the observed time at 7.1 to 7.4. The maximum TCID₅₀ titer achieved was 2.4×10⁷ virus/mL, the maximum HA titer was 2.2 log HA-units/100 μl.

The results of this growth experiment show that no glucose limitation occurred due to the feeding of pyruvate, and thus the viable cell concentration does not collapse.

### EXAMPLE 6

### Virus Adaptation to CAP-1D5 Cells in PEM and 293SFMII Medium in 50 ml Falcons

The virus adaptation of influenza viruses A/Brisbane/59/2007 (H1N1-like HGR; IVR-148, NIBSC, The National Institute for Biological Standards and Control), B/Florida/4/2006 (NIBSC, The National Institute for Biological Standards and Control), swine influenza (A/Swine (H1N2) Bakum/1832/00; IDT biologics) and equine influenza (A/equine 2 (H3N8); A/Newmarket/1/93; NIBSC, The National Institute for Biological Standards and Control) was carried out by infection of CAP-1D5 over 4 passages in 50 ml falcon container in PEM and 293SFMII medium. Prior to each infection, medium was changed. The virus yield during each passage was quantified by titration of the hemagglutinin (log HA units/100 μl) and by the Tissue Culture Infectious Dose₅₀ Assay (TCID₅₀ virus count/ml), (Genzel and Reichl, Vaccine production—state of the art and future needs in upstream processing in Methods in Biotechnology. Animal Cell Biotechnology—Methods and Protocols, Ed R. Partner, Humana Press Inc., Totowa, N.J., 2007, 457-473; Kalbfuss et al, 2008; Biologicals 36 (3):145-61).

The results are presented in FIG. 5. The infection of the CAP-1D5 cells with the influenza virus A/Brisbane/59/2007 and B/Florida/4/2006 both in PEM, as well as in the 293SFMII medium led to significantly increased virus titers after 4 passages, wherein the increase of the virus titer in 293SFMII medium was stronger. Similar increase showed the titration of the HA value during infection of the CAP-1D5 cells with the influenza virus or A/Brisbane/59/2007 and B/Florida/4/2006, respectively, both in PEM and also in the 293SFMII medium. The infection of the CAP-1D5 cells with the swine influenza virus leads to an increase of the titration of the HA value, both in PEM, as well as in the 293SFMII medium.

Along with an increase in the virus titer via the adaptation, the replication of the virus became faster with each passage and the virus titer could ultimately be increased significantly. Generally it appears that a slight increase in the virus titers compared to PEM medium can be achieved in the 293SFMII medium.

### EXAMPLE 7

### Cultivation Experiments with the Permanent Amniocyte Cell Line CAP-1D5 at Increased Initial Cell Concentration

The permanent amniocyte cell line CAP-1D5 was cultivated in 100 ml of PEM medium (Invitrogen) at 37° C., 8% CO₂ and 185 rpm. The initial cell concentration was 5×10⁵ cells/ml and 8×10⁵ cells/ml, respectively. Additionally, pyruvate was added in the batches with an increased initial cell concentration at a final concentration of 4 mM and 10 mM, respectively and also other amino acids were added.

At time 0 (=start of the culture), and in each case in a period of 24 h, the viable cell concentration, the dead cell concentration, the survival rate of the cell line, as well as the pH value, the concentration of glucose, lactose, glutamine, ammonium, glutamic acid and pyruvate in medium (biochemical multiparameter analysis system) was determined (Lohr et al, Vaccine, 2009, 27 (36) 4975-4982; Genzel et al, Appl Microbial Biotechnol, 2010, 88 (2):461-75).

The results are presented in FIG. 6. By increasing the initial cell concentration from about 5×10⁵ cells per ml of culture at the beginning of the culture to 8×10⁵ cells per ml culture of the permanent amniocyte cell line CAP-1D5 in PEM medium, there was an additional yield of 1×10⁶ cells per ml of culture after 90 h. At the typical time of infection (about 96 h to 120 h after the start of the culture), a cell concentration of 5−6×10⁶ cells per ml culture was thus reached.

The pH value of the culture was at the typical time of infection (about 96 h to 120 h after start of the culture), in a rather critical range at 6.6 to 6.8. Preferably, the pH value at infection ought to be at about 7.2-7.4.

### EXAMPLE 8

### Effects of Variation of Trypsin Activity and Performance of a Medium Change and a 1:2 Dilution with Medium, Respectively, to the Virus Titer

This experiment was to investigate how the use of different trypsin concentrations in the virus infection, and a medium change, or a 1:2 dilution of the medium, will affect the virus titer.

The permanent amniocyte cell line CAP-1D5 was cultured in 100 ml of PEM medium (Invitrogen) with 4 mM pyruvate and glutamine at 37° C., 8% CO₂ and 185 rpm in shake flasks. The cell line was infected at the start time of the culture with CAP-1D5 cells adapted A/PR/8/34 influenza virus (H1N1, RKI, Robert Koch Institute). The number of cells in the culture was at the time of infection 4.5×10⁶ cells/ml culture medium, if no medium change took place prior to infection, and 2.3×10⁶ cells/ml of culture, respectively, if a 1:2 dilution with PEM medium took place before infection, and 5×10⁶ cells/ml, if a complete medium change was done prior to infection. Further, the cultures without a medium change and the cultures with a 1:2 dilution with PEM medium, used trypsin concentrations of 1×10⁻⁴ U/cell, 3×10⁻⁵ U/cell, and 5×10⁻⁵ U/cell was used. In the cultures with complete medium change, trypsin concentrations of 1×10⁻⁴ U/cell, 1×10⁻⁵ U/cell, 5×10⁻⁵ U/cell and 1×10⁻⁶ U/cell or no trypsin was used.

The results are presented in FIG. 7. The 1:2 dilution with fresh PEM medium leads to an early HA increase (about 12 h instead of 24 h) and higher maximum rates of HA-2.70 log HA compared to 2.30 log HA of the cultures that had no medium change. A complete medium change led to increased HA values that exceed 3.0 log HA.

In cultures without a medium change and with a 1:2 dilution with the PEM medium, very similar values were shown on the log HA values, regardless of the trypsin concentration. In the cultures, in which a complete medium change was carried out, the course of the log HA values is very similar at the trypsin concentrations of 1×10⁻⁵ U/cell, 5×10⁻⁵ U/cell and 1×10⁻⁶ U/cell. In the culture without trypsin, the log HA value reached only a value of about 2 log HA-units/100 μA and in the culture in which 1×10⁴ U/cell trypsin was used for the infection, the log HA value was below 1 log HA-unit/100 μl.

### EXAMPLE 9

### MOI (Multiplicity of Infection) Dependence of the Infection of CAP-1D5 Cells in PEM Medium with Different Adapted Influenza Virus Strains, with and without the Performance of a Medium Change Prior to Infection

With the present experiment, the dependence between the MOI value, i.e. the numerical ratio of the number of the virus particles used for infection and the number of CAP-1D5 cells to be infected and the virus titer (in log HA units per 100 μl of culture) were examined.

Therefore, the permanent amniocyte cell line CAP-1D5 was cultivated in 50 ml of PEM medium (Invitrogen) with each 4 mM pyruvate and glutamine at 37° C., 8% CO₂ and 185 rpm, in shake flasks. The MOI-dependence was tested both with and without a medium change of the culture prior to infection. Three different adapted influenza strains were used: A/PR/8/34 (RKI, Robert Koch Institute), A/Brisbane/59/2007 (IVR-148, NIBSC, The National Institute for Biological Standards and Control) and B/Florida/4/2006 (NIBSC, The National Institute for Biological Standards and Control). The infection occurred in 50 mL shake flasks with a cell concentration at inoculation of 4.9×10⁶ cells/mL (without medium change) and. 5.0×10⁶ cells/mL (with medium change).The infection was carried out at MOI values of 0.25 and 0.10 (for A/Brisbane influenza virus), 0.025 and 0.0025, if no medium change was performed and at MOI values of 0.10 and 0.06 (at A/Brisbane influenza virus), 0.025 and 0.0025, if a medium change was performed. Without a medium change, the trypsin activity was 1×10⁴ U/cell and with medium change, it was 1×10⁶ U/cell. Subsequently, the amount of virus particles over 144 hours was determined (log HA-Units/100μ) (Kalbfuss et al, 2008; Biologicals 36 (3):145-61).

The results are presented in FIG. 8. The performance of a medium change leads to more consistent results in the virus replication. A low MOI dependence can be seen only in cells infected with influenza virus A/PR/8/34 without medium change, and in cells infected with influenza virus A/Brisbane with medium change. With medium change, considerably higher log HA values may be reached. The pH values without medium change were partially in the critical range of 6.6 to 6.8, and at medium change in the range from 7.3 to 7.5.

### EXAMPLE 10

### Further Cultivation in 1 L Scale in STR and Wave Bioreactors with Infection (A/PR/8/34 Adapted)

In one approach (B16) CAP-1D5 cells were cultivated in a 1 liter bioreactor STR (Sartorius) in PEM medium with 4 mM glutamine and 4 mM pyruvate at 120 rpm, pH=7.2, and an oxygen partial pressure pO₂ of 40% with pure oxygen. The initial cell concentration was 5×10⁵ cells/mL. After 72.75 h growth and a cell concentration of 2.1×10⁶ cells/ml, the CAP-1D5 cells were infected with influenza virus A/PR/8/34 (adapted: in PEM, 4^{th} passage, 2.01×106 viruses/mL). No medium change was made. The MOI value was 0.025, and it was added trypsin in a final concentration 3×10⁻⁵ U/mL.

In another approach (B26) CAP-1D5 cells were cultivated in a 1 liter bioreactor STR (Sartorius) in PEM medium at 120 rpm, pH=7.4 to 7.2 and an oxygen partial pressure of pO₂ of 40% with pure oxygen. The initial cell concentration was 8×10⁵ cells/mL. After 92 h of growth, the CAP-1D5 cells (adapted: in PEM, 4^{th} passage, 3.75×10⁶ viruses/ml) were infected with influenza virus A/PR/8/34. Previously, a complete medium change was made, and the pH value adjusted to 7.6. The MOI value was 0.025, and trypsin in a final concentration of 3×10⁻⁵ U/mL was added.

In a third approach (wave) CAP-1D5 cells were cultured in a 1 liter bioreactor Wave (Wave Biotech) of PEM medium with 4 mM glutamine, 4 mM pyruvate and 20 mM glucose at a rocking frequency of 13 rpm, at an angle of 7°, pH=7.3 to 6.9 and an oxygen partial pressure pO₂ of 40% with pure oxygen and a partial pressure of CO₂ of 7.5%. The initial cell concentration was 5×10⁵ cells/mL. After 72 h of growth, the CAP-1D5 cells (adapted: in PEM, 4th passage, 1.87×10⁶ cells/ml) were infected with influenza virus A/PR/8/34. The cell concentration before infection was 2.1×10⁶ cells/ml. No medium change was made. The MOI value was 0.025, and trypsin was added in a final concentration of 3×10⁻⁵ U/mL.

In a fourth approach MDCK.SUS2 cells were cultured in the 1 liter bioreactor STR (Sartorius) in AEM medium. The initial cell concentration was 5×10⁵ cells/mL. After 118.25 hours of growth the cells at MDCK.SUS2 were infected with influenza virus A/PR/8/34 (Lohr et al., Vaccine, 2010, 28 (38):6256-64).

The results are presented in FIG. 9. Over a period of 192 h the viable cell concentration and dead cell concentration was determined, as well as the pH value, the concentration of glucose, lactose, glutamine, ammonium, glutamic acid and pyruvate in the medium. Further, from the time of infection, the log HA-units/100 μl and TCID₅₀ values were detected (Genzel and Reichl, Vaccine production determined—state of the art and future needs in upstream processing in Methods in Biotechnology: Animal Cell Biotechnology—Methods and Protocols, Eds R. Partner; Humana Press Inc., Totowa, N.J., 2007, 457-473; Kalbfuss et al, 2008; Biologicals 36 (3):145-61).

The CAP-1D5 cells grow faster in all three approaches and in higher density as compared with MDCK.SUS2 cells. The virus titers in the CAP-1D5 cell cultures reach a value for the log HA-units/100 μl of about 2.5 at maximum. The virus titer of the cell culture MDCK.SUS2 reaches a maximum value for the log HA-units/100 μl of about 3. The virus titers in the CAP-1D5-cell cultures increase much earlier, compared with the virus titer in the cell culture at MDCK.SUS2.

### EXAMPLE 11

### Cultivation Experiment to Increase the Virus Yield in Shake Flasks in PEM or 293SFMII Medium with Complete Medium Change or 1:1 Medium Change Prior to Infection

To optimize the yield of virus in CAP-1D5-cell cultures, which are cultivated in shake flasks the CAP-1D5 cells were cultured in the media 293SFMII (Invitrogen) and PEM (Invitrogen) and a 1:1 media change or a complete media change was performed.

The permanent amniocyte cell line CAP-1D5 was cultured in 50 ml of PEM medium with 4 mM glutamine and 4 mM pyruvate at 37° C., 8% CO₂ and 100 rpm in 100 ml shake flasks. Prior to infection of the cells with adapted influenza virus A/PR/8/34 at an MOI of 0.025, a medium change was performed. If a 1:1 medium change was performed, trypsin at a concentration of 1×10⁻⁵ U/cell was used for the infection of the cells. If a complete medium change was made, trypsin at a concentration of 1×10⁻⁶ U/cell was used for the infection of the cells. The medium change occurs with both PEM medium, as well as with 293SFMII medium. The cell concentration at infection was 5×10⁶ cells/ml.

The results are presented in FIG. 10. Over a period of 72 h, the viable cell concentration, the survival rate, the pH value, as well as the log HA-units/100 μl were determined by titration of hemagglutinin using a standard method (Lohr et al., Vaccine, 2009, 27 (36), 4975-4982; Genzel et al, Appl Microbiol Biotechnol, 2010, 88 (2):461-75).

The virus titer increased faster in those cell cultures, in which a complete medium change was carried out before infection with the influenza virus A/PR/8/34 than in the cell cultures, in which a 1:1 medium change was performed. Besides, the virus titer in the cell cultures, in which prior to the infection with influenza virus A/PR/8/34, a complete medium change was performed, reached a higher maximum virus titer than in those cell cultures in which a 1:1 medium change was performed.

The viable cell concentration of the cell cultures with complete medium change before infection decreased from 5×10⁶ cells/ml after 24 h, so that it was about at 2×10⁴ cells/ml after 48 h. The cell culture with PEM medium and 1:1 medium change before infection decreased least strongly. In this case the viable cell concentration after 72 h was still about 7×10⁵ cells/ml.

The pH value in all cell cultures during the entire period of time of 72 h was between 7.6 and 7.2.

1-15. (canceled)

16. A method for the production of an influenza virus based vaccine, comprising
a) infecting a permanent human amniocyte cell with an influenza virus;
b) culturing the permanent human amniocyte cell;
c) expression of the influenza virus; and
d) isolating the influenza virus from the medium,
wherein the permanent human amniocyte cell expresses the adenoviral gene products E1A and E1B.

17. The method according to claim 16, wherein the permanent human amniocyte cell is in or between the exponential growth phase and the stationary growth phase at the time of infecting with the influenza virus.

18. The method according to claim 16, wherein the isolation of the influenza virus from the medium in step d) takes place by means of density gradient differential or zonal centrifugation.

19. The method according to claim 16, wherein the adenoviral gene products E1A and E1B comprise the nucleotides 1 to 4344, 505 to 3522 or the nucleotides 505 to 4079 of the human adenovirus serotype-5.

20. The method according to claim 16, wherein the permanent human amniocyte cell expresses the adenoviral gene product pIX.

21. The method according to claim 16, wherein a complete medium change or a 1:2 dilution with medium takes place prior to the infection with an influenza virus.

22. The method according to claim 16, wherein a trypsin concentration of 1×10⁻⁴ U/cell, 1×10⁻⁵ U/cell, 3×10⁻⁵ U/cell, 5×10⁻⁵ U/cell or 1×10⁻⁶ U/cell is added when infecting with an influenza virus.

23. The method according to claim 16, wherein a virus amount indicated as MOI value in the range of 0.001 to 0.3 is used, when infecting with an influenza virus.

24. The method according to claim 16, wherein the influenza virus is a human influenza virus, an equine influenza virus or a swine influenza virus.

25. The method according to claim 16, wherein the influenza virus is selected from the group consisting of influenza virus strains A/PR/8/34, A/Uruguay/716/2007, A/Brisbane/59/2007, B/Florida/4/2006, swine influenza (A/Swine (H1N2) Bakum/1832/00) and equine influenza (A/Equine, A/Newmarket/1/93 (H3N8)).
